(19) United States
(12) Patent Application Publication
(10) Pub. No.: US 2026/0013851 A1
(43) Pub. Date: Jan. 15, 2026

(54) MINIMALLY-INVASIVE ENDOSCOPIC SUTURING DEVICE

(71) Applicant: ANCHORA MEDICAL LTD., Caesarea (IL)

(72) Inventors: Jonatan EPSTEIN, Ramat Hasharon (IL); Avraham Rami LORE, Kiryat Tiv'on (IL); Rotem BAAVOUR, Regavim (IL); Yuval GONEN, Kiryat Tiv’on (IL); Gonen SOMEKH, Kerem Maharal (IL); Omer RAVID, Kfar Yona (IL); Boaz HARARI, Ganey Tikva (IL); Ariel HELLER, Gan HaShomron (IL); Shirel DICOVSKY, Netanya (IL)

(21) Appl. No.: 18/996,610

(22) PCT Filed: Jul. 19, 2023
(86) PCT No.: PCT/IL2023/050759

§ 371 Date: Jan. 17, 2025

Related U.S. Application Data
(60) Provisional application No. 63/390,367, filed on Jul. 19, 2022.

(51) Int. Cl. A61B 17/04; A61B 17/00; A61B 17/06

(52) U.S. Cl. CPC A61B 17/0469; A61B 17/0401; A61B 17/06066; A61B 2017/00818; A61B 2017/0409; A61B 2017/0496

(57) ABSTRACT

An apparatus comprising: a tubular grasper comprising resilient tines that are configured to expand outwardly when unbiased; a needle disposed inside the tubular grasper, and configured to penetrate tissue; a tubular anchor disposed on the needle; and a surgical thread threaded through the tubular anchor.

### CROSS-REFERENCE TO RELATED APPLICATIONS

This application claims priority to U.S. Provisional Patent Application No. 63/390,367, filed Jul. 19, 2022, entitled “Minimally-Invasive Endoscopic Suturing Device,” the contents of which are incorporated herein by reference.

### FIELD OF THE INVENTION

The invention relates to the field of endoscopic surgical tools.

### BACKGROUND

Suturing remains a common approach for repair of live tissue and is used for tissue closure, approximation, ligation, and fixation of tissue access sites, organs, vessels, fixation of meshes and other implants or devices and the like. Although largely dependent on the skill of the surgeon or endoscopist, the results obtained using a suture are highly predictable and reliable.

Alternatives to suturing that were developed over the years, such as clips, staples, fasteners (also known as “tacks”), anchors, and tissue adhesives, have gained varying degrees of acceptance and are used for tissue repair in both open and minimally-invasive procedures, such as laparoscopy and flexible gastrointestinal (GI) endoscopic procedures.

Still, manual suturing remains a mainstay of surgical repair, but not without disadvantages. Manually forming a multitude of stitches can be tiring and time-consuming, which can lead to suturing errors compromising the integrity of repair. In fact, for many procedures the time spent suturing may be greater than the time spent treating the underlying target tissues. In endoscopic procedures, manual suturing is almost impossible, and the various suturing assistive devices available on the market tend to be cumbersome and difficult to use. Even robotic surgical platforms, while able to suture very accurately and delicately, are typically limited by the speed the remote-operating surgeon can manipulate the robotic platform's handles.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

Some embodiments are directed to an apparatus for suturing tissue, comprising: a flexible elongated shaft configured to be inserted through a work channel of an endoscope; a handle disposed at a proximal end of said elongated shaft; multiple anchors disposed in a single file inside said elongated shaft, wherein each of said multiple anchors comprises: an elongated body, and an extension emerging outwardly from said elongated body; a thread disposed along the length of said elongated shaft and threaded sequentially through said extensions of said anchors; a grasping mechanism triggerable by said handle and being configured to secure the tissue to the apparatus during deployment of the anchors; and an anchor deployment mechanism triggerable by said handle and being configured to: penetrate through the tissue with said elongated shaft or with a needle disposed inside said elongated shaft, and deploy said anchors, one by one, from said elongated shaft or the needle into a space beyond the tissue, such that, when said thread is tensioned, a longitudinal dimension of each deployed anchor lies flush with a surface of the tissue.

In some embodiments, said grasping mechanism comprises a plurality of sharp elements configured to penetrate the tissue axially, or a helix configured to be threaded into the tissue.

Some embodiments are directed to an apparatus for suturing tissue, configured to grasp the tissue from a first side thereof, deploy multiple anchors at the opposite side of the tissue, and optionally tightly secure a suture that is threaded through said anchors and is disposed mostly at the first side of the tissue.

Some embodiments are directed to a method for minimally-invasive, full thickness suturing.

Some embodiments are directed to a method for minimally-invasive gastrointestinal surgery.

In some embodiments, there is provided an apparatus comprising: a tubular grasper comprising resilient tines that are configured to expand outwardly when unbiased; a needle disposed inside the tubular grasper, and configured to penetrate tissue; a tubular anchor disposed on the needle; and a surgical thread threaded through the tubular anchor.

In some embodiments, there is provided a method comprising: (a) inserting a flexible endoscope through a body orifice of a patient, the flexible endoscope comprising a work channel having a distal opening; (b) providing a tubular grasper which comprises resilient tines that are configured to expand outwardly when unbiased; (c) exposing the tubular grasper from the distal opening of the work channel, and unbiasing the tines such that the tines penetrate and secure a tissue as they expand outwardly; (d) providing a needle that is disposed inside the tubular grasper, and a tubular anchor that is disposed on and secured to the needle, and wherein a surgical thread is threaded through the tubular anchor; (c) pulling the tubular grasper proximally, such that the secured tissue forms a sleeve, the tissue is punctured by the needle, and the tubular anchor passes to the distal side of the tissue while still secured to the needle; (f) withdrawing the needle proximally and releasing the tubular anchor from the flexible needle at the distal side of the tissue, within the formed sleeve; (g) biasing the tines of the tubular grasper such that the tissue is released from the tines; (h) loading a new tubular anchor onto the flexible needle, and repeating steps c, e, f, and g with respect to the new tubular anchor; (i) tensioning the surgical thread so as to form a suture extending between the tubular anchor and the new tubular anchor; and (j) securing the surgical thread relative to the new tubular anchor, such that the tension is maintained.

In some embodiments, the apparatus further comprises one or more actuators configured to: push the tubular grasper distally, out of a distal end of an endoscope, such that the tines expand outwardly, penetrate tissue, and secure the tissue to the grasper; pull the tubular grasper proximally, such that the tissue that is secured to the grasper is pulled proximally while the needle and the tubular anchor penetrate the tissue so as to position the anchor at the distal side of the tissue; pull the flexible needle proximally and release the tubular anchor beyond the tissue; and tension the surgical thread at the proximal side of the tissue, thereby causing the tubular anchor to pull the tissue proximally.

In some embodiments, the one or more actuators are comprised at least partially in a handle.

In some embodiments, the tubular anchor comprises a tubular body and a resilient extension that is configured to expand outwardly from the tubular body when unbiased, and wherein the extension comprises a ring through which the surgical thread is threaded.

In some embodiments, the apparatus further comprises: an inner shaft to which the tubular grasper is affixed; and a flexible, tubular, outer shaft configured to enclose the inner shaft.

In some embodiments, the one or more actuators are comprised at least partially in a handle, and wherein the inner shaft extends to the handle.

In some embodiments, the apparatus further comprises: a tubular grasper collector mounted over the outer shaft and configured to enclose the tubular grasper.

In some embodiments, each of the tines of the tubular grasper has a blunt edge.

In some embodiments, the extension is a cut through the wall of the tubular anchor.

In some embodiments, a length ratio between the tubular body of the anchor and the extension of the anchor is between 1:0.3 and 1:0.7.

In some embodiments, the tubular body of the anchor has a chamfered distal edge to facilitate penetration of the tissue together with the needle.

In some embodiments, the tubular body of the anchor has a beveled distal edge to facilitate penetration of the tissue together with the needle.

In some embodiments, there is provided a tissue grasper comprising: a tubular body made of a resilient material and having a plurality of tines configured to expand outwardly when unbiased; a tube configured to enclose the tubular body and the tines and to bias the tines such that they assume a tubular configuration; and an actuator configured to: (a) push the tubular body relatively to the tube, such that the tines exit the tube and penetrate a tissue while expanding outwardly, so as to secure the tissue to the tines, and (b) pull the tubular body relatively to the tube, such that the tines enter the tube while releasing the tissue.

In some embodiments, the resilient material is a superelastic metal alloy, and wherein the tines are trained to a normally-expanded configuration.

In some embodiments, the plurality of tines are 3-8 tines.

In some embodiments, the plurality of tines are 4-7 tines.

In some embodiments, the plurality of tines are 5-6 tines.

In some embodiments, each of the tines has a blunt edge.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-1B show perspective views of an apparatus for suturing tissue.

FIG. 1C shows a perspective view of an alternative apparatus for suturing tissue.

FIGS. 2A-2B show a side view and a perspective view of a distal portion of a shaft with a grasper.

FIGS. 2C-2F show perspective views of alternative configurations of grasping means, each disposed side-by-side with a needle.

FIGS. 3A-3H show various views of different anchors.

FIG. 4 shows a cross-sectional view of two anchors attached to tissue.

FIG. 5 shows a cross-sectional view of a distal portion of an apparatus for suturing tissue disposed inside an endoscope.

FIGS. 6A-6F show cross-sectional views of multiple stages of deploying anchors into tissue.

FIGS. 7A-7B show a perspective view and a cross-sectional view of a guide shaft for separate advancement and deployment of anchors and their extensions.

FIG. 8 shows a perspective view of distal portion of an alternative apparatus for suturing tissue, in which a needle is housed within a jawed grasper.

FIG. 9 shows a perspective view of a slitted needle.

FIGS. 10A-10B show perspective views of whorls which serve as grasping means.

FIGS. 11A-11B show top isometric views of two whorls.

FIGS. 12A-12B show cross-sectional views of a sleeve arrangement.

FIG. 12C shows a perspective view of a distal portion of the sleeve arrangement of FIGS. 12A-12B.

FIGS. 13A and 13B show perspective views of an anchor.

FIGS. 13C and 13D show a perspective view and a side view of another anchor.

FIGS. 14A and 14B show perspective views of a typical push operation which takes place using a ratchet mechanism of the sleeve arrangement of FIGS. 12A-12B.

FIGS. 15A-15C show a shaft arrangement for use with the sleeve arrangement of FIGS. 12A-14C.

FIG. 15D shows a cross-sectional view of the sleeve arrangement of FIGS. 12A-12B with the shaft arrangement of FIGS. 15A-15C.

FIGS. 15E-15F show perspective views of a grasper.

FIG. 15G shows a cross-sectional view of a tine penetrating the stomach wall.

FIGS. 15H-15M, which show perspective views of six alternative configurations of a grasper.

FIGS. 16A-16D are cross-sectional views illustrating four stages of implanting an anchor beyond tissue.

FIG. 17A is a photograph of a porcine experiment performed with a tined grasper.

FIG. 17B is a photograph of another porcine experiment with a tined grasper.

FIGS. 18A and 18B shows a perspective view and a cross-sectional view, respectively, of a distal area of an alternative shaft arrangement.

FIGS. 19A-19C show a side view and two perspective views, respectively, of an alternative grasper in a closed configuration.

FIGS. 20A-20C show a side view and two perspective views, respectively, of the alternative grasper in an open configuration.

FIGS. 21A and 21B show a top view and a side view, respectively, of a distal area of one of tines of the alternative grasper.

FIG. 21C shows a side view of one of the tines of the alternative grasper in an expanded state.

FIG. 22 shows a plan view of a sheet of material from which the alternative grasper is fabricated.

FIGS. 23A-23C show a side view, a front view, and an enlarged view of a needle.

FIGS. 25A-25D show side views of an alternative anchor in a closed configuration.

FIGS. 26A-26C show side views of the alternative anchor in an open configuration.

FIG. 27 shows a side view of another anchor in an open configuration.

FIG. 28 shows a flow chart of a method for suturing tissue.

### DETAILED DESCRIPTION

Disclosed herein is an apparatus for suturing tissue through a flexible endoscope, and a method for minimally-invasive suturing of tissue using the apparatus. The apparatus may be used for various endoscopic procedures requiring full-thickness suturing of soft tissue, in order to close, approximate, fixate, plicate, and/or ligate the tissue.

For example, the apparatus may be used, through a gastroscope or a colonoscope, for performing a variety of upper and lower GI (Gastrointestinal) procedures in a minimally-invasive manner, namely—from an esophageal or rectal approach. Exemplary procedures include original sleeve gastroplasty or revision to an existing sleeve gastroplasty, fistula closure, endoscopic submucosal dissection (ESD), endoscopic mucosal resection (EMR), per-oral endoscopic myotomy (POEM), gastric bypass revision, stent or other prosthetic fixation, closure or prevention of perforation, closure or prevention of bleeding, lumen apposition, and others.

Advantageously, pertinent parts of the apparatus may be sized to fit within a single work channel (also “working channel,” “instrument channel,” etc.) of an endoscope, thereby leaving one or more other work channels available for other tools needed during the procedure, such as various imaging, illumination, suction, irrigation, and/or tissue manipulation tools. The work channel may have an inner diameter of, for example, 2-8 millimeters (mm), and the apparatus may have its relevant parts (e.g., its outermost shaft) sized and configured to fit within that space.

Advantageously, the apparatus may deploy multiple anchors threaded over a suture (also called “thread”); the apparatus, anchors, and suture are all configured in a way that: (a) prevents or mitigates frictional damage to surrounding tissue, (b) allows the tissue to be closed and/or approximated by a convenient and simple pulling of a thread, after the anchors have been deployed satisfactorily, (c) enables strong tissue fixation, (d) reduces tension on the sutured tissue (compared with regular, manual suturing) and thus reduces the risk of tissue trauma and thread ‘cheese cuts’, (e) does not form a loop that encapsulates tissue, and thus decreases the risk for nerve or blood vessel entrapment, (f) enforces standardized suturing thus reduces the variability between surgeons/endoscopists of different skill levels, (g) divides suture tension between all anchors (h) focuses force applied to the tissue on the interface between the tissue and each of the anchors, which have a much larger surface area then a surgical thread (also simply referred to as a “suture”), and/or (i) enables simple, actuator-operated suturing even in remote anatomical locations, with minimal expertise required.

In the following description, the described functionality and use of the various components constitutes a method for suturing tissue, even if the terms “method” and “steps” are not explicitly mentioned.

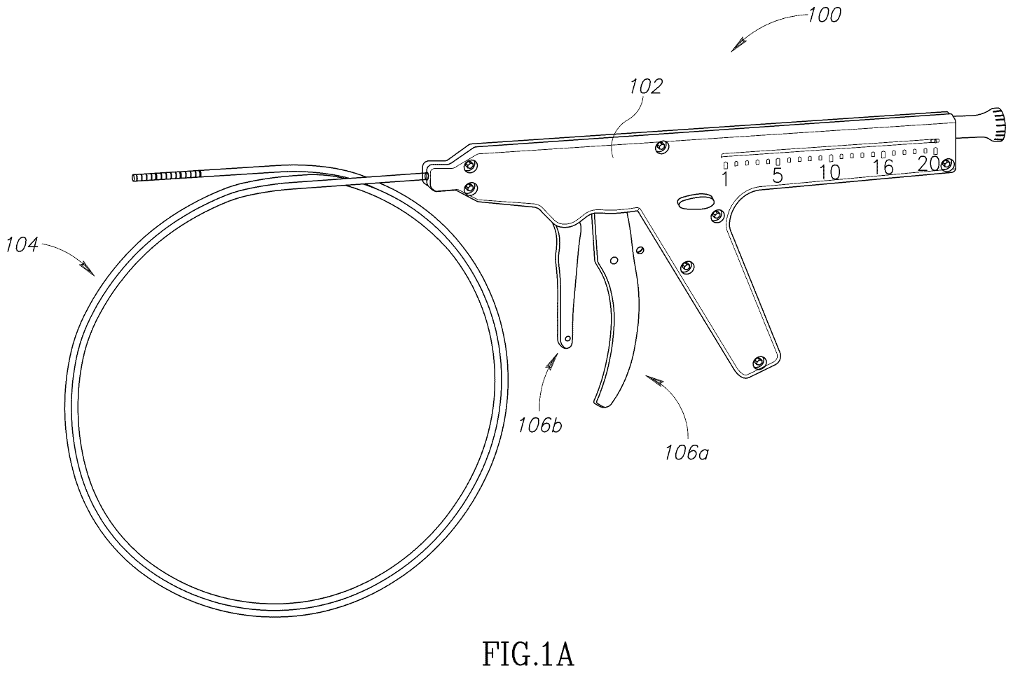

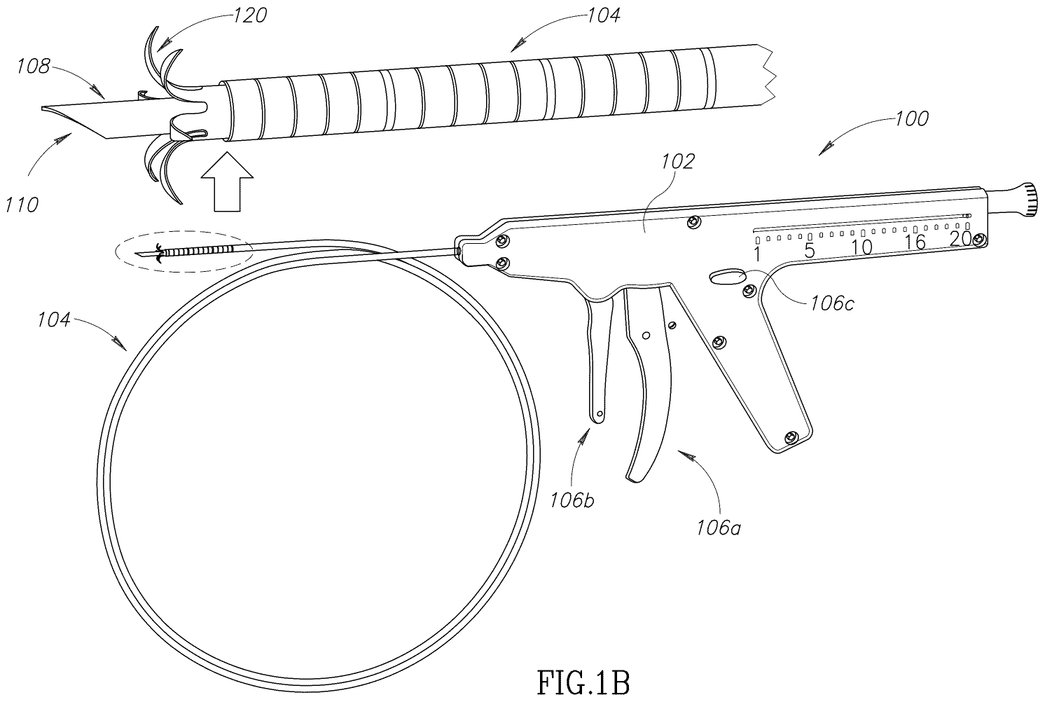

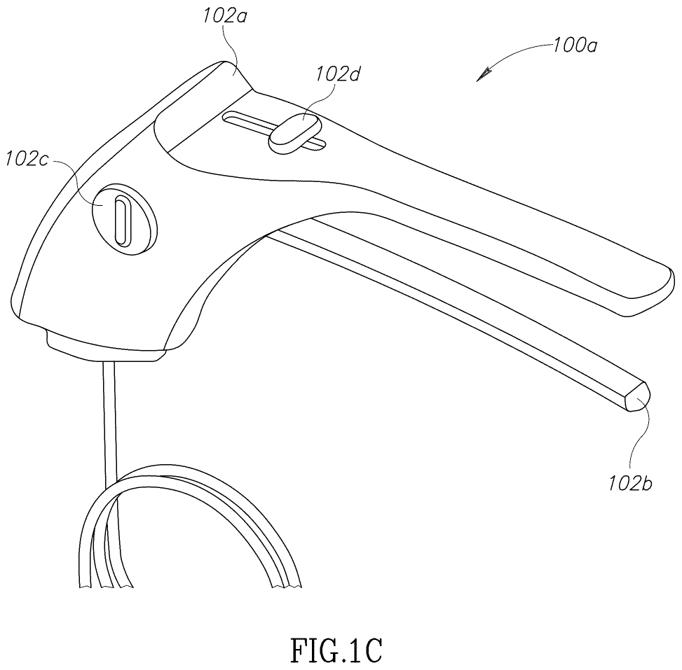

Reference is now made to FIGS. 1A and 1B, which show perspective views of an apparatus 100 for suturing tissue, in accordance with an embodiment. Apparatus 100 may include a handle 102, and a flexible shaft 104 extending from a distal end of the handle.

Shaft 104 may be a flexible elongated tube, made, for example, of plastic and/or braided metal. Shaft 104 may have a length of, for example, 200-3000 mm, wherein part of this length, such as between 10-300 mm, may be disposed inside handle 102. Shaft 104 may have an external diameter of, for example, 2 to 10 mm. Shaft 104 may have a uniform or a variable diameter along its length.

A needle 108 may be disposed inside shaft 104, and optionally has a beveled edge 110. Alternatively, a needle (not shown) may have a straight, non-beveled edge. Needle 108 is shown protruding distally from shaft 104 in the view of FIG. 1B. Needle 108 may be used to penetrate tissue, in order to deploy anchors beyond the tissue. Needle 108 may have an external diameter of, for example, 0.5 to 9.8 mm, and a wall thickness of, for example, 0.1 to 0.6 mm. Needle 108 may be made of stainless steel or any other suitable material, such as Nitinol (nickel-titanium alloy). Optionally, needle 108 has an internal anchor stopper (not shown) near its distal end (e.g., between 1-20 mm from the distal end), embodied as a resilient protrusion from an inner wall of the needle into the lumen of the needle-thus decreasing the inner diameter of the needle at that location. The stopper may normally protrude into the lumen of needle 108, but may be pushed completely or partially out of the lumen when an anchor (further discussed below) applies on it a sufficient amount of force. The stopper may be constructed such that it can resist a predetermined amount of force.

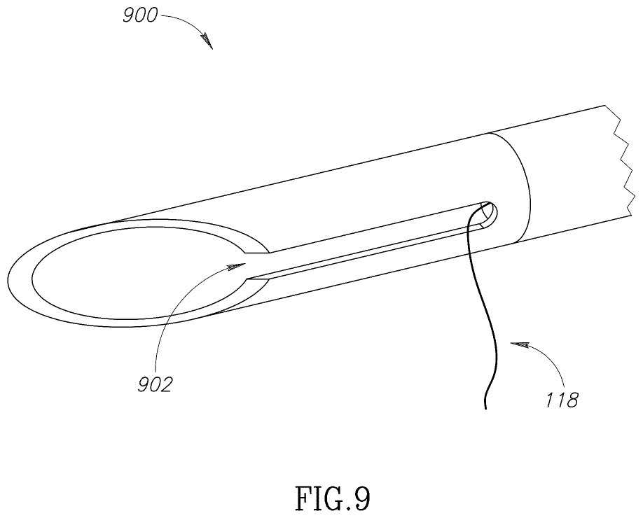

Interim reference is made to FIG. 9, which shows an optional embodiment of a needle 900, which has a slit 902 at its distal end. Slit 902 may be configured to contain suture 118 as the apparatus is moved between one anchor deployment location to another, and may prevent severing of the suture as it chafes against the needle's distal end. Slit 902 may be configured with smooth edges that do not sever the suture when it chafes against them, as opposed to a needle without such a slit, such as needle 108 of FIGS. 1A-1B whose distal end may include sharp edges that can sever the suture. Slit 902 may have a length of, for example, 1-30 mm, and a width of, for example, 0.4-2 mm.

Back to FIGS. 1A-1B, a grasper 120 may also be disposed inside shaft 104, for example over needle 108, and be used to grasp the tissue during tissue puncturing with the needle, and deployment of the anchors. Without such grasping means, an attempt to pierce the tissue just with a needle may actually push the tissue, especially if it is very soft, flexible, or flailing, away from the needle. Although the needle might finally pierce the tissue, this may occur only after substantial extension of the needle in the distal direction, which might cause injury to an unintended organ, vessel, nerve, or other tissue. Accordingly, it is beneficial to grasp and secure the tissue to shaft 104 during it piercing by the grasper.

Optionally, grasper 120 and shaft 104 are configured to move distally or proximally over or in needle 108. Needle 108 can optionally be static in relation to handle 102, and thus, once grasper 120 has grasped the tissue, the grasper can be withdrawn back in relation to the needle, keeping the needle in place and at the same location and distance from the tissue (as well as from any vessels, nerves, or other organs), thus enabling safe tissue piercing.

Optionally, shaft 104 can extend over and cover needle 108 and/or gasper 120 while in the endoscope or in the anatomical cavity, shielding the endoscope or the anatomical cavity, respectively, from damage or injury. Shaft 104 may be made of plastic, metal, and/or metal alloy, and be configured with sufficient flexibility along its longitudinal axis so that it can comply with the flexing of the endoscope containing it.

Grasper 120 is shown protruding distally from shaft 104 in the view of FIG. 1B. Grasper 120 is structured as a tube which is separated at its distal portion into multiple sharp grasping tines (e.g., between 2 and 10 tines, or more than that) that penetrate the tissue. Grasper 120 may be made of a shape-memory alloy, such as Nitinol, and the grasping tines may be trained to an expanded configuration in which they curl away from the central axis of the grasper, and optionally even backwards. While grasper 120 is still inside shaft 104, the grasping tines are biased to a contracted configuration, in which they arrange flatly around needle 108. As grasper 120 is expelled distally and the grasping tines exit the distal end of shaft 104, they curl outwardly and penetrate the tissue that faces the distal of the shaft.

FIG. 1C shows an apparatus 100a, which is a variant of apparatus 100 (of FIGS. 1A-1B) in which the handle 102a is structured differently. Handle 102a may include various actuators, such as a trigger 102b, a dial 102c, and a sliding button 102d, which are configured to control various functionalities of apparatus 100a, such as manipulation of its shaft, needle, grasper, anchor pusher, and/or the like. The term “actuators,” however, should not be interpreted as limited to such elements on handle 102a; instead, an actuator may be any physical device that is connected to one or more part (e.g., shaft, needle, grasper, anchor, pusher, inner shaft, outer shaft) of an apparatus (such as, but not limited to, apparatus 100a), and configured to move and/or maintain that part stationary.

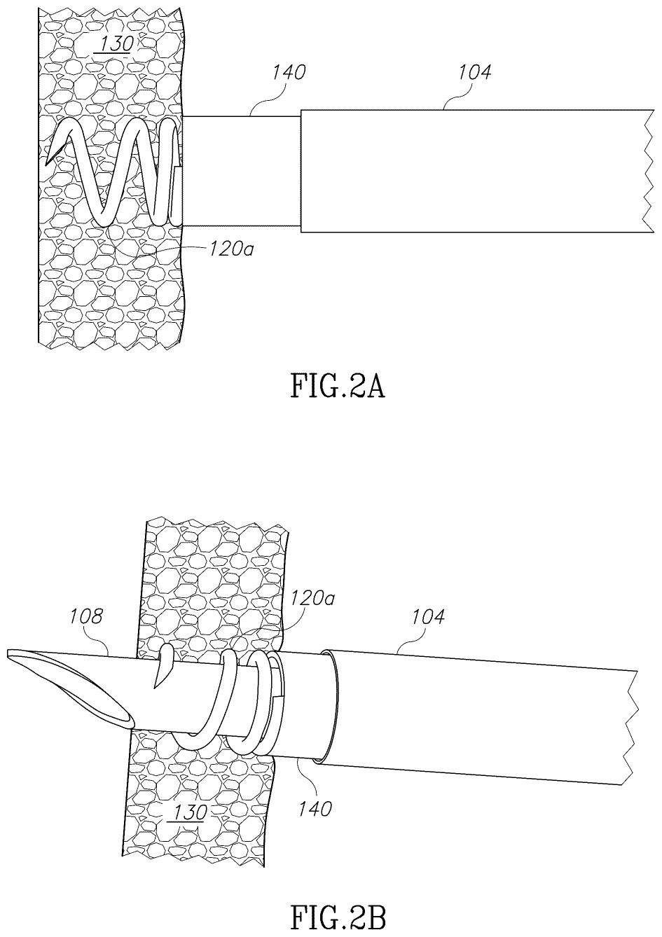

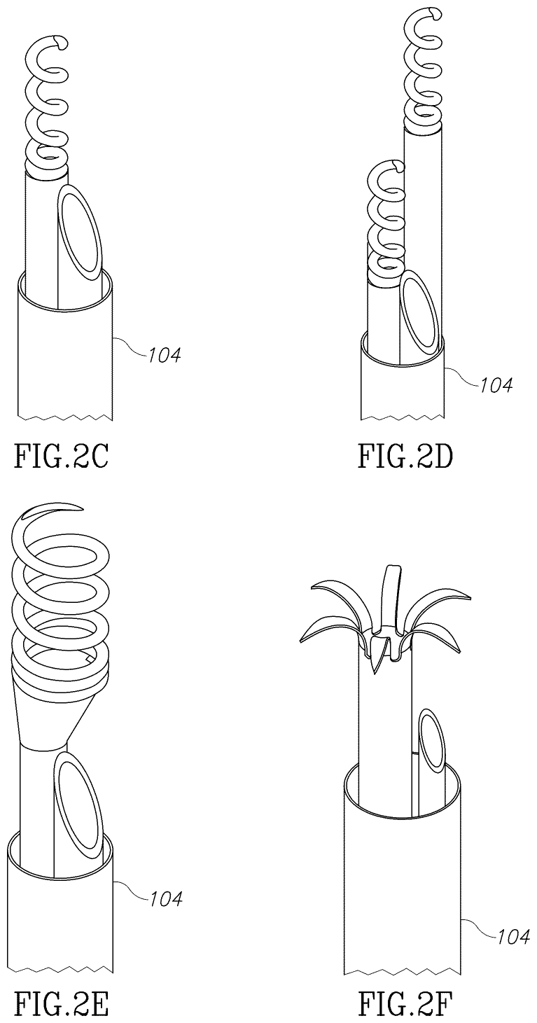

Reference is made to FIGS. 2A-2B, which show schematic illustrations of a distal portion of a shaft 104 with an alternative grasper embodiment, structured as a helix 120a. Helix 120a may be attached to, connected to, or integrally formed with a distal end of a hollow extension shaft 140, which may rotatably push and withdraw the helix into and out of the tissue. Namely, helix 120a may be screwed into tissue 130, to secure shaft 104 to the tissue. In FIG. 2A, helix 120a is shown extended from the distal end of shaft 104 (and needle 108, not seen, is withdrawn inside extension shaft 140), which is the configuration in which the helix is already screwed into the tissue 130. In FIG. 2B, needle 108 is shown extended through helix 120a and beyond it, which is the configuration in which the anchors are deployed at the opposite side of the tissue, from the opening of the needle.

Although helix 120a is shown with approximately three full rotations around the central axis of the helix (namely, about 1080 degrees), an alternative helix (not shown) may have anywhere between a quarter of a full rotation (90 degrees), or 20 full rotations. Also, while helix 120a is shown as a single helix, an alternative helix (not shown) may be a double helix or even a triple helix that are wound around the same central axis.

Reference is made to FIGS. 2C-2F, which schematically illustrate alternative configurations of grasping means, each disposed side-by-side with a needle (instead of over it), inside shaft 104.

In FIG. 2C, a single helix is shown side-by-side with a needle.

In FIG. 2D, two helices are shown side-by-side with a needle.

In FIG. 2E, an expandable helix is shown side-by-side with a needle. This helix may be made, for example, of a shape-memory alloy (such as Nitinol), trained to a larger diameter than the radial space it occupies inside shaft 104, next to the needle. When the helix exits shaft 104, it expands to its larger diameter, and is therefore able to better grasp the tissue compared to, for example, the helix of FIG. 2C (assuming a shaft and a needle of the same measurements). Optionally, this helix may be threaded into the tissue concurrently with its gradual advancement out of shaft 104, so that it gradually stretches the tissue during the threading. The advantages of stretching the tissue in addition to grasping it are discussed below, with reference to a whorl-shaped grasper.

In FIG. 2F, a grasper configured with tines, similar to grasper 120, is shown side-by-side with a needle.

Optionally, apparatus 100 includes a helix protection sleeve (not shown), configured as a tube around helix 120a but inside shaft 104. This sleeve may be advanced distally to cover any segment of helix 120a that is disposed beyond needle 108 (e.g., beyond the proximal end of its bevel) and is not within tissue; that segment may be prone to entanglement of suture 118 between the wounds of the helix, and the sleeve thus acts to physically separate the suture and the helix and prevent or reduce the chances of entanglement.

Any of the helices discussed above may be made, for example, of metal, metal alloy, or plastic, and configured to be sufficiently strong for being threaded through the tissue without breakage or substantial plastic deformation.

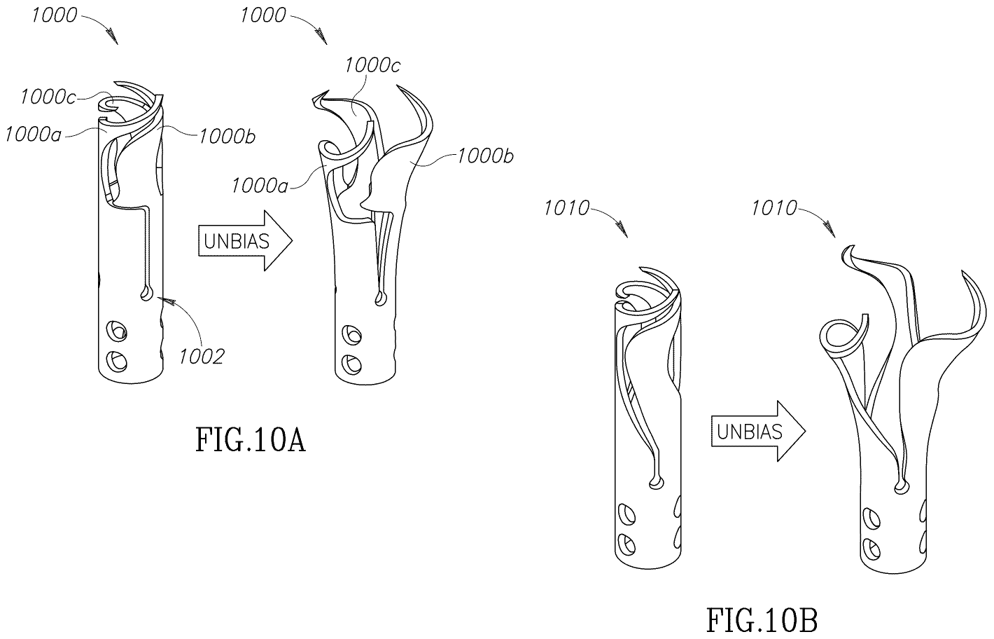

Reference is made to FIG. 10A which shows yet another grasper embodiment, structured as a whorl 1000. Whorl 1000 may have a basic tubular shape, with an inner diameter configured to allow the whorl to be mounted over needle 108, and an outer diameter configured to allow it to be disposed inside shaft 104. The tubular wall of whorl 1000 may include multiple cutouts (such as three cutouts, as shown in this example) extending generally downwardly from the upper end of the tubular wall, and forming a respective number of helical, pointed leaves therebetween, such as three leaves 1000a-c. A greater number of leaves is also possible, such as between 2 and 7 leaves. Each of the cutouts may terminate proximally with a smooth, optionally circular end 1002, which is configured for housing the suture (if it happens to pass between the leaves during any part of the procedure) without severing it.

The left side of FIG. 10A shows whorl 1000 in a retracted configuration, in which leaves 1000a-c define a substantially uniform tubular shape, while the right side of the figure shows whorl 1000 in an expanded configuration, in which the leaves protrude radially. Whorl 1000 may be made of a shape-memory alloy (such as Nitinol) trained to the expanded configuration, such that the whorl can be biased to the retracted configuration while inside shaft 104, and expand upon becoming unbiased-when exiting the shaft.

In operation, shaft 104 may be brought into contact or proximity with the tissue, followed by pushing whorl 1000 distally while rotating it. The pointed edges of leaves 1000a-c then pin into the tissue and gradually stretch it as the leaves continue to expand and rotate. Namely, whorl 1000 may both grasp the tissue and stretch it. Stretching the tissue may make it thinner within the stretched region, thereby requiring lesser distal movement of needle 108 in order to fully penetrate the thickness of the tissue. In addition, when the tissue is stretched from two or more points (the pointed edges of the leaves), it will tend to remain substantially parallel to these points and hence substantially parallel to the distal opening in shaft 104; this will ensure that needle 108 can penetrate the tissue substantially perpendicularly to the tissue, and lower the risk of the needle approaching the tissue at a blunt angle and not penetrating it well enough or not at all. In addition, this may contribute to needing less force to push the needle to pierce the tissue, and/or to enabling the use of a less sharp needle with the same pushing force (thus lowering the risk of injury to other organs or tissues from the needle's sharp end).

Following anchor deployment and needle retraction, the tissue may be released by pulling whorl 1000 back into shaft while rotating the whorl in the opposite direction. This will gradually return the tissue to its resting state, while eventually disengaging the pointed edges of leaves 1000a-c from the tissue. Alternatively, needle 108 remains static and whorl 1000 advances distally from shaft 104, over the needle. Whorl 1000 may be rotated to be released from the tissue, as described above, and retreated into shaft 104. While retreating, the leaves will gradually return to their retracted configuration, assuming a generally tubular shape together.

Reference is now made to FIG. 10B which shows an alternative whorl 1010, in which the cutouts in the tubular body are shaped differently than in FIG. 10A, and hence form pointed leaves of a different shape. This is intended to illustrate that generally, a whorl-type grasper may be configured with such leaves that allow desired levels of tissue penetration and tissue stretching, in correspondence with a desired extent of distal pushing of the whorl and of rotating it. For example, the pitch angle of the helical upper area of the leaves may affect a tradeoff between tissue penetration and required rotation: a smaller pitch angle will generally penetrate the tissue at a blunt angle and grasp it more strongly per a certain depth of penetration, but require more rotation to achieve that depth, while a larger pitch angle will generally penetrate the tissue at a sharp angle and grasp it less strongly per that certain depth of penetration, requiring less rotation to achieve that depth. As another example, the extent of radial expansion of the leaves when unbiased may affect the amount of stretching the tissue will experience per a certain length of distal pushing of the whorl: more radial expansion generally means less distal pushing to achieve the same level of stretching. There is also an interplay between the radial expansion of the leaves when unbiased and the pitch angle of the helical upper area of the leaves.

Generally, the pitch angle and/or radial expansion may be adapted for the suturing task at hand, such as the thickness, pliability, and resistance to penetration of the tissue involved.

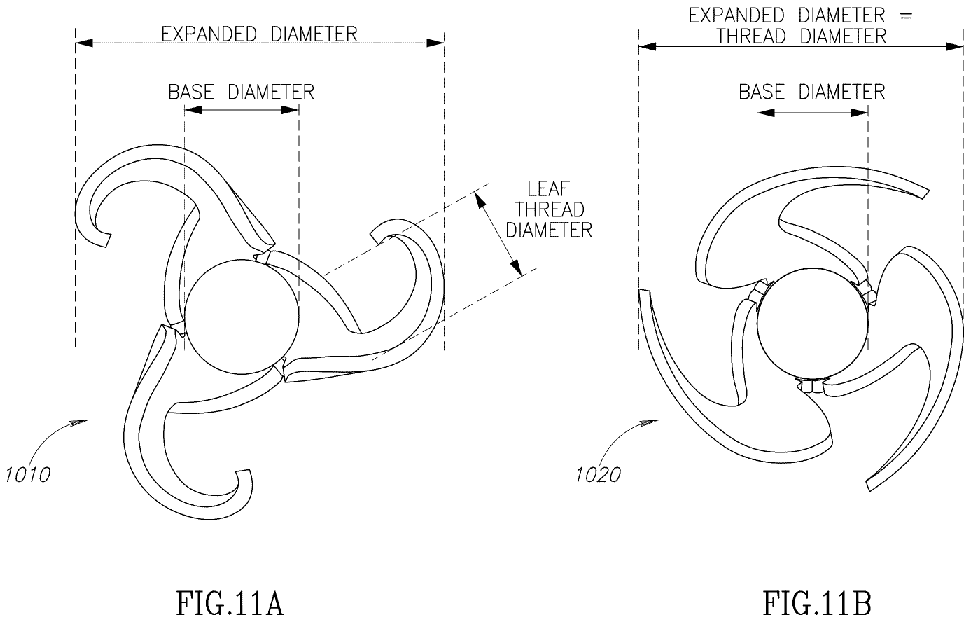

Reference is now made to FIGS. 11A-11B, which are top isometric views of whorl 1010 and a different whorl 1020, in their expanded configurations, demonstrating various parameters of possible embodiments of a whorl. For purposes of demonstration, the bases of these two whorls have equal internal diameters. However, their leaves are configured differently, which influences the manner these leaves thread into tissue and stretch the tissue.

First, the leaves, when expanded, may extend to a certain overall diameter, which is larger for whorl 1010 and smaller for whorl 1020. This expanded diameter may be derived from the training of the shape-memory alloy and/or from the shape to which the leaves are cut. The expanded diameter may affect the amount of stretching the tissue will undergo, but is not the only parameter involved in the stretching, as is further explained below.

Second, the posture of the expanded leaves may define the diameter of the circular threading of the leaves into the tissue. In FIG. 11B, the leaves of whorl 1020 assume, together, a substantially circular shape, defining a thread diameter which is substantially the same as the expanded diameter of the leaves. When whorl 1020 is gradually expanded as it is threaded into tissue, it may gradually stretch the tissue in an attempt to assume its unbiased, expanded diameter. In FIG. 11A, however, the leaves of whorl 1010, at their edges, curl inwardly from the expanded diameter, each forming its own smaller thread diameter. When this whorl 1010 is threaded into tissue while expanding, its stretching of the tissue may be different than that achieved with whorl 1020.

Another relevant parameter may be wall thickness of the whorl, at least along its regions which are trained to bend. This parameter may affect the expansion force exerted by the whorl. Generally, thicker walls of a shape-memory alloy whorl may endow the whorl with stronger expansion force, and vice versa. This, in turn, may affect the amount of tissue stretching that the whorl can achieve.

Accordingly, different whorl embodiments may each have a different combination of such parameters, and all such possible combinations are explicitly intended herein.

The base diameter may measure, for example, 0.5-15 mm. The expanded diameter may measure, for example, 0.8-30 mm. The individual leaf thread diameter, if different from the expanded diameter, may measure, for example, 0.5-29 mm. The wall thickness, at least along the whorl's regions which are trained to bend (and optionally along the entire whorl) may measure, for example, 0.1-4 mm.

Throughout this disclose, generic reference to a “grasper” or even to “grasper 120” is intended to also refer to helix 120a or to whorl 1000/1010, mutatis mutandis.

The terms “pushing,” “pulling,” “advancing,” etc. are used herein to describe relative motion of two or more parts of an apparatus. For example, when “advancing” of a needle is described, the needle may either be physically manipulated to move forward relative to the handle, or the needle may remain static relative to the handle but one or more other parts of the apparatus may be withdrawn backwards relative to the needle so that it assumes a more forward position. The same rationale applies to other parts of the apparatus.

With reference back to FIGS. 1A-1C, handle 102 may include one or more user-operable actuators, such as, but not limited to, one or more triggers 106a and/or 106b and a button 106c. These user-operable actuators may be configured to and serve to manipulate shaft 104, needle 108, grasper 120, and/or to deploy anchors. For example, trigger 106a may be used to push grasper 120 out of the distal end of shaft, to cause the grasper to engage the tissue. Button 106c may be used to retract grasper 120 back into shaft 104 simultaneously with advancing needle 108 into and through the tissue. Trigger 106b may be used to push the anchors one by one from the distal end of needle 108; for example, each full swing of trigger 106b backwards may push the single file of anchors by a length equal to one anchor (or a similar length). These are only examples of the functionality of user-operable actuators included with the handle. In other embodiments, various user-operable actuators may be differently configured.

Multiple anchors may be disposed in a single file inside needle 108 (or over the needle—in a configuration not shown in the figures). Optionally, a spacer may be disposed between every two adjacent anchors, to ensure that when one anchor is extracted from needle 108, the anchor behind it does not protrude from the distal end of the needle. The spacers may be threaded over suture 118 or not. They may be made of a bioabsorbable material so that they will be absorbed and consumed in the body. Alternatively, if the suturing operation is of the gastrointestinal tract, the apparatus may be configured such that the spacers are left, after deployment of the anchors, inside the lumen of the esophagus, stomach, or intestine, so that they are later naturally expelled from the body with other fecal matter. Optionally, the spacers may help prevent unintentional retraction of an ejected anchor back into needle 108.

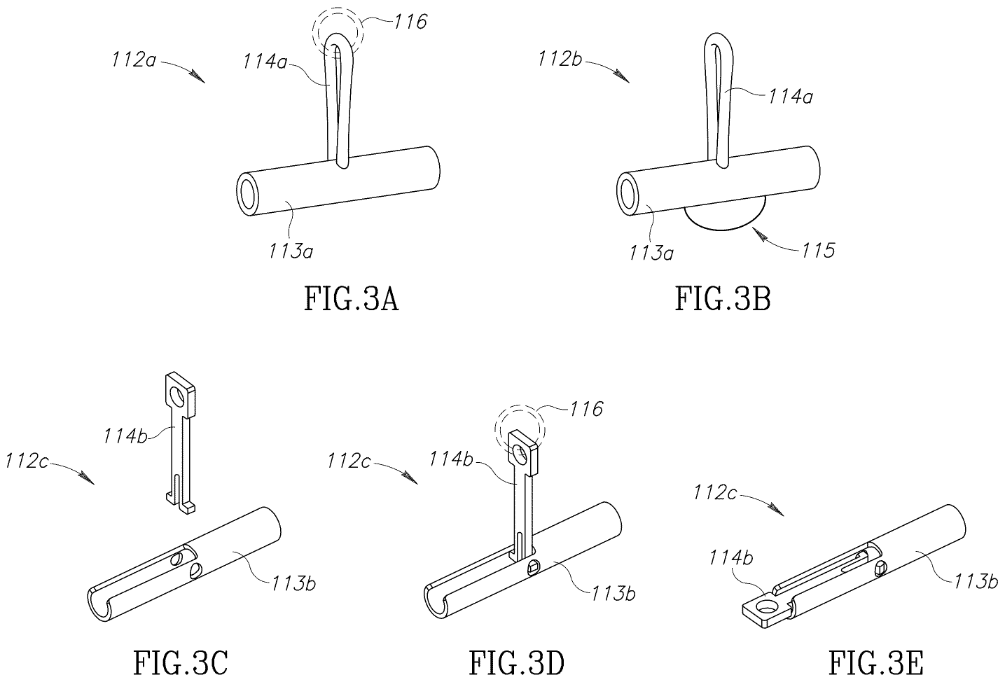

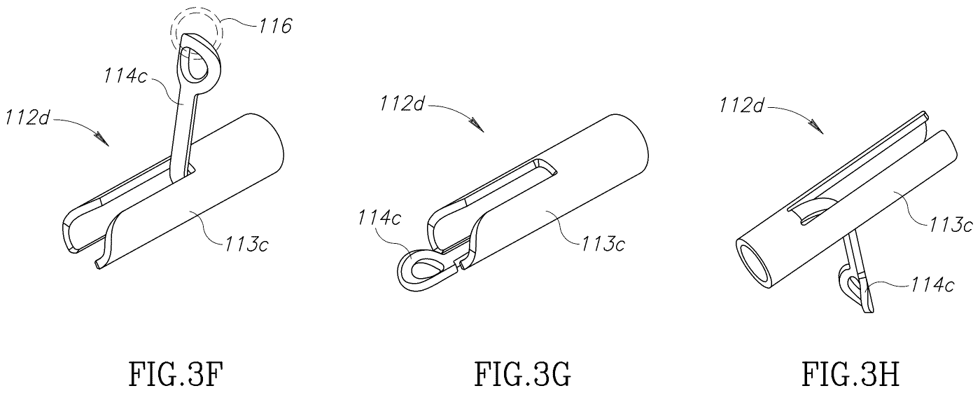

Exemplary anchors 112a-112d (jointly referred to hereinbelow as “anchor 112”) are shown in FIGS. 3A-3H. FIG. 3A shows a first exemplary anchor 112a. FIG. 3B shows a second exemplary anchor 112b. FIGS. 3C-3E show a third exemplary anchor 112c in an exploded view, an expanded configuration, and a retracted configuration. FIGS. 3F-3H show a fourth exemplary anchor 112c in an expanded configuration, a retracted configuration, and a bottom view of the expanded configuration.

Each of anchors 112 may have an elongated body 113a-c, hollow or solid, made of a rigid or flexible material, such as stainless steel, Nitinol, titanium, and/or plastic such as polyetheretherketone (PEEK), (permanent or bioabsorbable). The elongated body may have a maximal external diameter of, for example, 0.5-5 mm, and a length of, for example, 2-15 mm. If hollow, its wall thickness may be between 0.1-1 mm, for example.

A flexible or rigid extension may emerge from anchor 112. As shown in FIGS. 3A-3B, the extension may be, for example, a cord 114a made of a surgical thread (sometimes referred to as a “surgical suture”) or be a flexible metal wire.

As shown in FIGS. 3C-3H, the extension may be a flexible or rigid rod 114b-c (straight, curved, spring-coiled, etc.) made of a suitable material such as one of the materials mentioned with respect to the anchor body.

Any of extensions 114a-c is optionally also tensile along its longitudinal axis. Each of extensions 114a-c may have a circular profile or a non-circular profile. Each of extensions 114a-c may be attached to, connected to, or integrally formed with anchor 112. Each of extensions 114a-c may have a diameter or a width (as the case may be) of, for example, 0.1-2 mm, which may be uniform or variable along its length.

Optionally, any of extensions 114a-c is formed as a resilient element, which is perpendicularly (or substantially perpendicularly, such as forming an angle of between 60-90 degrees with the anchor) attached to, connected to, or integrally formed with anchor 112. This way, when anchor 112 is inside its containing lumen (e.g. needle 108 or shaft 104, in case the anchors are threaded over the needle), extension 114a-c is biased to lie parallel or substantially parallel to the anchor (e.g., between 150-180 degrees relative to the longitudinal axis of the anchor); this may increase friction between the cord and the internal wall of the needle, preventing accidental extraction of the anchor from the apparatus. Later, when anchor 112 is extracted from needle 108, extension 114a-c assumes its non-biased, perpendicular posture. This posture ensures that anchor 112 will lie flat or substantially flat against the distal side of the tissue once the suture is tensioned and secured.

Alternatively, any of extensions 114a-c is formed as a rigid element which is pivotably connected to anchor 112, so as to allow the extension to rotate between an expanded configuration (in which it erects perpendicularly or substantially perpendicularly from the anchor) and a retracted configuration (in which it lies parallel or substantially parallel with the anchor).

In FIGS. 3A-3B, body 113a of anchor 112a-b is similarly a hollow tube having a circular profile. Cord 114a forms a loop outside body 113a. Inside body 113a, two ends (not shown) of cord 114a may be tied or otherwise fastened together and/or separately fastened to the body.

FIG. 3B shows an anchor 112b mostly similar to anchor 112a of FIG. 3A, but with an optional functionality—one or more resilient wings 115 that are biased to lie flush with the anchor while it is inside needle 108, but deploy and expand once the anchor is extracted from the needle. Each resilient wing 115 may be an arcuate wire made of a superelastic material such as Nitinol, or an elastic material such as stainless steel, cobalt-chromium alloy (CoCr), or the like. Alternatively, a resilient wing (not shown) may be integrally formed with the anchor, such as a cutout in the anchor's wall that is trained to expand radially outwardly when unbiased. Wing 115, when expanded, makes anchor 112b wider than the diameter of needle 108 in at least one plane, which prevents anchor 112d from falling back into the needle after it has been extracted from it. In some embodiments, wing 115 may also provide friction between anchor 112b and the inner wall of needle 108, such that the foremost anchor 112b in the needle does not get extracted accidentally. Although wing 115 is shown in conjunction with anchor 112b which is otherwise similar to anchor 112a, it may just as well be implemented with any of anchors 112b-d or with any other anchor discussed herein but not illustrated. Furthermore, the functionality of anchor expansion after its extraction from the apparatus, and/or anchor friction with its containing lumen (e.g., needle), may be realized differently than the wing shown. For example, it may be realized by at least one resilient extension that is configured to lie flatly or substantially flat against the anchor when biased, and expand to a distance of 0.5 to 5 mm from the anchor when extracted from the containing lumen.

In FIGS. 3C-3E, anchor 112c has a tubular body 113b with a longitudinal cutout from one end of the tubular body to approximately the middle of its length, or slightly beyond the middle. A tab 114b is pivotably connected to body 113b, by a pair of protrusions at one end of the tab, which fit into matching apertures in the body's wall. An aperture is disposed at or near the opposing end of tab 114b, for threading of a suture 118 through the tab. When anchor 112c is disposed inside needle 108, tab 114b is maintained fully or partially retracted inside body 113b. After anchor 112c is ejected from needle 108, tab 114b may be rotated to assume a perpendicular or substantially perpendicular posture with respect to body 113b. Optionally, anchor 112c (or any of the other anchors) may include an extension locking mechanism (not shown) that is configured to lock the extension in a perpendicular or substantially perpendicular position relative to the anchor's body. For example, that locking mechanism may be configured to self-lock the extension once it reaches the perpendicular or substantially perpendicular position.

In FIGS. 3F-3H, anchor 112d has a tubular body 113b with a tab 114c which, differently from FIGS. 3C-3E, is cut from the tubular body itself. Rotation of this tab 114c is by flexing the material of tubular body 113b at the area where the tab begins. This flexing may be plastic deformation or elastic deformation, depending on the material of which anchor 112d is made. An aperture is disposed at or near the opposing end of tab 114c, for threading of suture 118 through the tab. Anchor 112d may be made, for example, of a shape-memory alloy (such as Nitinol), trained with tab 114c bent to a perpendicular or substantially perpendicular posture with respect to body 113b. This way, tab 114c can be biased to a retracted configuration when inside needle 108, and will spring back to its trained posture upon its extraction from the needle-thereby preventing anchor 112d from accidentally being pulled back into the needle.

In a further configuration of an anchor (not shown), the extension may be realized by a single-strand cord extending from the anchor's body, and having a ring tied or attached to it at its opposing end.

Optionally, the aperture, ring, or loop of any of anchors 112a-d is smooth and lacks any sharp edges, thus preventing or mitigating damage to both suture 118 and the tissue from friction with the aperture, ring, or loop. Such smoothing may be achieved by a suitable surface treatment and/or by coating at least the inner surface of the aperture, ring, or loop with a smooth material, optionally polymeric. Optionally, an additional ring is connected to the extension of any of the above-described anchors, such as a toroidal, smooth ring 116 shown with broken lines in FIGS. 3A, 3D, and 3F. Suture 118 may be threaded through ring 116 instead of through the extension, and the smoothness of the ring's surface may further reduce friction and prevent damage to the suture. In an alternative configuration (not shown), a low-friction ring may not be toroidal but rather have a different shape that is still devoid of sharp edges that may damage the suture.

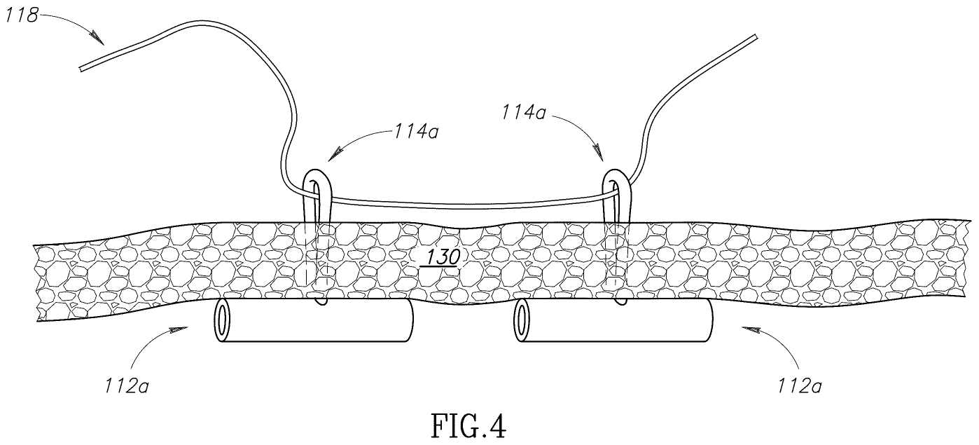

FIG. 4 shows two of anchors 112a attached to tissue 130, with cords 114a penetrating fully through the tissue and connecting a suture 118. Anchors 112a may be attached to tissue 130 such that cords 114a impale the tissue to protrude from its opposite side. Suture 118 may run through the loops of cords 114a at the opposite side of tissue 130, such that, when the suture is pulled tight, the portions of the cords that protrude from the tissue can bend to prevent damage to the tissue. Anchors 112b-d of FIGS. 3B-3H may function similarly.

To eject anchors 112 one by one from shaft 104, apparatus 100 may include a pusher, such as a rod (not shown) disposed inside needle 108 that pushes the single file of anchors from behind the most proximal anchor.

As an alternative to pushing anchors 112 all at once from behind, to affect ejection of the most distal anchor, apparatus 100 may include a different pusher (not shown) that is configured to push only the most distal anchor. For example, such pusher may be disposed over (or inside) the single row of anchors, without contacting all but the most distal anchor. The contact between a distal end of this pusher and the most distal anchor may be such that pushing of that anchor distally may be facilitated by advancing the pusher distally, to a distance sufficient to expel the anchor from needle 108. Then, the pusher may be retracted backward, so that it can push the next anchor when desired.

Reference is now made back to FIGS. 1A-1B. Suture 118, which is not seen in these figures, may be disposed inside needle 108 along its length, and optionally exiting a distal end of the needle. Suture 118 may be threaded sequentially through the rings or loops of the anchors that are disposed serially inside needle 108. Suture 118, at its proximal area, is optionally wound around a spool (not shown) disposed inside handle 102.

Suture 118 is optionally a surgical thread (sometimes referred to as a “surgical suture”), which may be bioabsorbable or non-bioabsorbable. Suitable bioabsorbable materials include, for example, polyglycolic acid, polylactic acid, monocryl, and polydioxanone. Suitable non-bioabsorbable materials include, for example, nylon, polyester, PVDF (Polyvinylidene fluoride), and polypropylene. Suture 118 may be a braided thread, a monofilament thread, or a multifilament thread. Suture 118 be made of metal or plastic, or of any biocompatible material. Suture 118 may be tensile or substantially non-tensile.

Suture 118 is optionally secured, at one end, to the spool at the handle, and extends from there through the loops or rings of all the anchors sequentially. The other end of suture 118 may either remain unattached, or be attached (e.g., with a knot, adhesive, cinch, etc.) to the most distal anchor. The latter means that, after a series of anchors are deployed, suture 118 may be cut at a location proximal the last-deployed anchor, and only the new proximal end of suture 118 has to be pulled in order to tighten the suture that extends through the deployed anchors; that new proximal end may then be secured to the last anchor deployed, either with a manual knot or with a cinch that prevents the suture from sliding through the ring or loop of that anchor.

Optionally, the attachment of suture 118 to the most distal anchor may be unidirectional, such as by a large knot of the suture over itself that is made distally to that anchor, so that the anchor cannot slide further distally but rather only proximally. Further optionally, after two or more anchors have been deployed and their suture cut, tensioned and secured, apparatus 100 may undergo preparation for a further suturing operation (assuming that at least two anchors remain in the apparatus) by making a knot of the suture (the suture remaining inside the apparatus, of course) distally to the now most-distal anchor inside shaft 104, that enabling the same manner of tensioning and securing as discussed above.

Optionally, apparatus 100 includes a suture severing mechanism, such as a sharp blade (not shown), for cutting suture 118 at a location proximal to the last deployed anchor.

Advantageously, the fact the suture is not threaded through the bodies of the anchors but rather through the rings or loops that are distanced from the anchor bodies and are not implanted into tissue, prevents the suture from applying force to the tissue, which may, in extreme cases, even cut the tissue.

Furthermore, when the suture is tensioned to finally close the tissue, the distancing of the rings or loops from the anchor bodies reduces stress from the anchor bodies and concentrates that stress at the rings or loops, and slightly along the cords.

Further yet, the use of anchors means that there is a greater implanted surface area, which can oppose the forces attempting to extract the anchors from the tissue. If only a suture were used, the only surface area securing the suturing were that the suture itself—which is very little. Mucosal tissue (such as that of the stomach) in particular is known to be challenging to suture. It does not usually hold well with sutures or clips. Moreover, high-tension suturing of such tissue will often slice the tissue, and even if the tissue manages to maintain its integrity—the sutures may occasionally cause ischemia and necrosis. Accordingly, suturing mucosal tissue (and other types of soft, easily-torn tissue) with the present anchors may overcome these problems, and allow durable tissue closure, approximation, or the like, without damaging the tissue. On one side of the tissue, the side wall of the anchor, along the anchor's longitudinal dimension, contacts the tissue and spreads the applied force over a large area; on the opposite side of the tissue, the force pulling the anchors towards the tissue is distributed across the entire suture that interconnects the rings or loops of all the anchors. Moreover, the tightening of the suture is performed in a friction-free environment—outside the soft tissue and through rings or loops whose inner surface is smooth and causes extremely little friction with the suture. This way, even during the patient's recovery period, the occasional rubbing of the suture over the surface of the rings or loops does not tear or otherwise degrade the suture.

After all anchors are implanted and the suture is tensioned, it may either be manually tied with a knot, or secured with a cinch (not shown). If both ends of the suture are free following the implantation, the cinch used may be a connector that mounts over two suture portions (or the suture portions be threaded through the cinch), not allowing them to move relative to one another. If the distal end of the suture has already been secured to the first-implanted anchor and only the proximal end of the suture remains free, the cinch used may be a device which mounts over the suture by friction and pushes against the ring of the last-implanted anchor.

The placing of the cinch over the two suture portions or the single suture portion may be performed using a separate tool manipulated by the surgeon (via the endoscope), or by a functionality in the apparatus itself, triggerable from the handle. The cinch is optionally one-directional, namely, once mounted on the one or two suture portions it can slide only distally but not proximally. This way, the sliding of the cinch contributes to the tensioning of the suture, and ensures that the tension cannot be accidentally loosened.

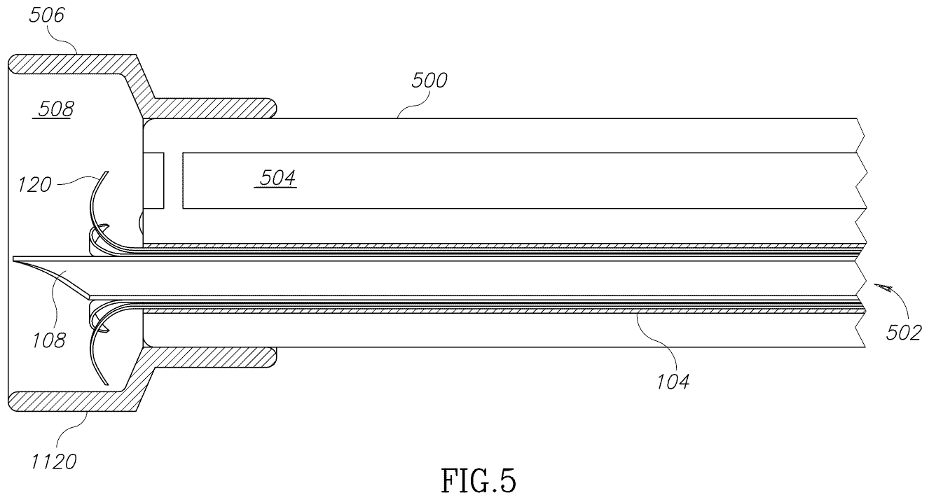

Reference is now made to FIG. 5, showing a cross-sectional schematic illustration of the distal portion of apparatus 100, whose shaft 104 is disposed inside a work channel 502 of an endoscope 500. One of the other channels 504 of endoscope 500 optionally serves as a suction lumen, in accordance with an embodiment. In that embodiment, a suction cup 506 may be mounted on a distal portion of the endoscope 500, to enable aspirating a larger tissue area into a void 508 of the suction cup. When suction is activated, the tissue is withdrawn into void 508 and towards the distal end of shaft 104. The distal opening of suction cup 506 may have a diameter of, for example, 10-30 mm. The effective depth of suction cup 506 (namely, the distance between its distal opening and the distal end of endoscope 500) may be between 3-20 mm, for example. The volume of void 508 may be, for example, between 1-40 cc (cubic centimeters).

Optionally, the suction also enables distancing the tissue from adjacent organs that may be injured from grasper 120 or the needle 108 if these accidentally penetrate also the adjacent organ. Optionally, once suction lumen 504 provides the suction, grasper 120 is used to more securely attach shaft 104 to the tissue. Optionally, once grasper 120 secures the tissue, the suction may be turned off until the next use. Optionally, the suction may be operated by a controller (not shown) configured on the handle 102 or as a foot-operated switch (not shown) or the like.

As an alternative to suction lumen 504, shaft 104 or needle 108 themselves may serve a suction lumen. Namely, shaft 104 or needle 108 may be connected to a source of suction at their proximal sides, to enable tissue aspiration at their distal sides.

In other embodiments, work channel 504 is not used for tissue aspiration and suction cup 506 is not present. Instead, work channel 504 (and optionally, one or more additional work channels) may be used, for example, for insertion of a camera, illuminator, optical fiber, and/or a surgical tool.

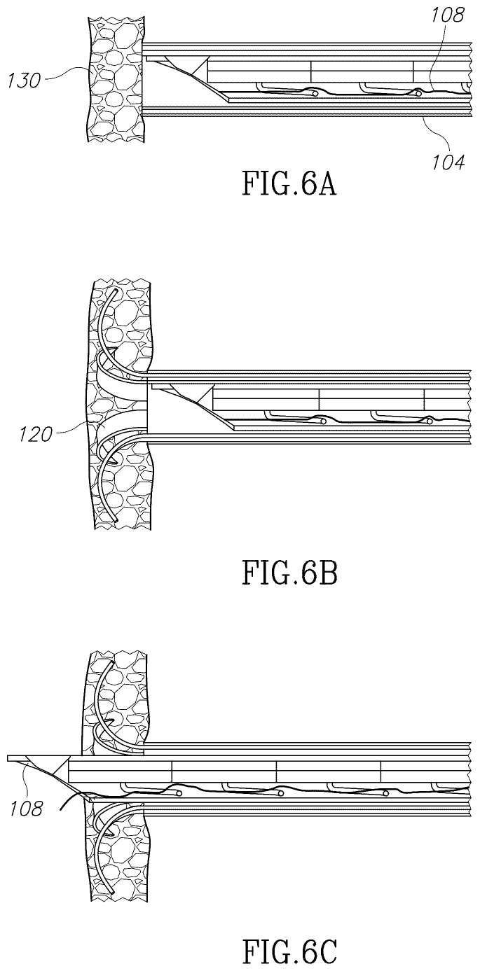

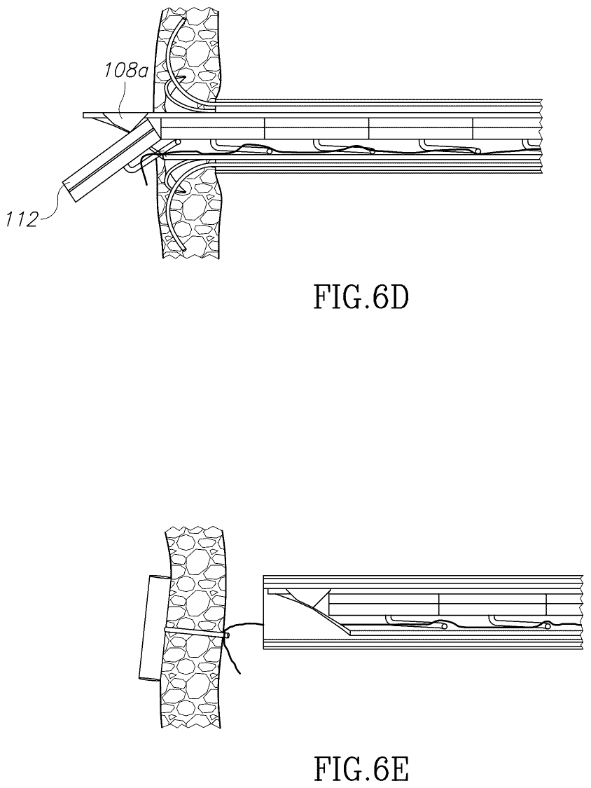

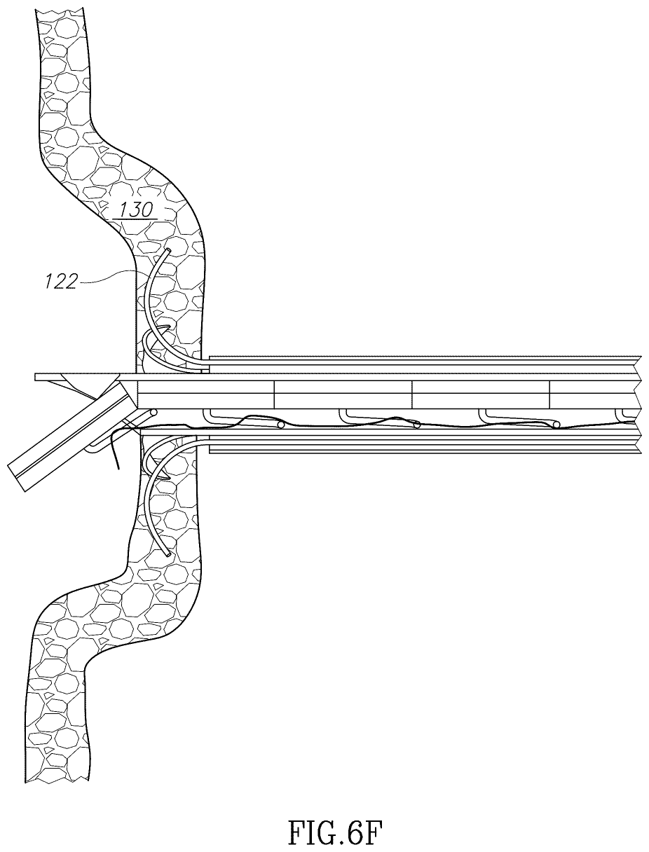

Reference is now made to FIGS. 6A-6E, showing cross-sectional views of multiple stages of deploying anchor 112 into tissue 130 with apparatus 100 of FIGS. 1A-1B, in accordance with an embodiment. For simplicity of illustration, an endoscope through which shaft 104 is inserted is not shown in these figures.

The distal end of shaft 104 of the apparatus may approach and contact the tissue 130 (FIG. 6A). For this, the endoscope (not shown) may be held in place while pushing shaft 104 slightly out of the endoscope. As shaft 104 is aligned with the tissue, grasper 120 may be extended out of shaft 104 and penetrate tissue 130 (FIG. 6B). Needle 108 is then extended out of shaft 104 through the secured tissue 130 (FIG. 6C). Anchor 112 may then be deployed via the needle 108 to the opposite side of tissue 130 (FIG. 6D). Finally, grasper 120 and needle 108 may be retracted into shaft 104 and the apparatus moved back (FIG. 6E), leaving the anchor inside tissue 130.

In FIGS. 6A-6E, suture 118 is shown freely threaded through the ring/loop of the distalmost anchor. This requires, after implanting a number of anchors required for the suturing task, to pull both sides of the suture to tighten it. In an alternative embodiment (not shown), the suture may be permanently affixed to that ring/loop, such as using a knot or a connector, so that final tightening of the suture only requires pulling its proximal side.

An optional feature shown in FIGS. 6A-6E is a deflector 108a disposed at or near the opening of needle 108. Deflector 108a may be a protrusion from the inner wall of needle 108, aimed at deflecting an ejected anchor in the opposite direction. For example, when deflector 108a is positioned at the upper side of needle 108, it will deflect each ejected anchor downwardly. The deflection tilts the ejected anchor relative to the central axis of needle 108 and shaft 104, so that, when these are withdrawn backwards, the anchor does not withdraw with them but rather remains at the opposite side of the tissue. Deflector 108a may be shaped as a wedge whose surface which contacts the anchor forms a sharp angle (e.g., between 10-60 degrees) with the central axis of needle 108 and shaft 104. Deflector 108a may also function as the stopper which was discussed above.

FIG. 6F shows a variant of the usage of apparatus 100, in which, after grasper 120 is secured to tissue 130, the apparatus (or at least its shaft) is pulled proximally together with the secured portion of the tissue. This creates space at the distal side of the tissue which is likely to be free of other tissues or organs, so that the extension of the needle and the deployment of the anchor does not damage these tissues/organs. For example, when using apparatus 100 to suture the stomach wall, pulling the wall before the needle penetrates it may lower the likelihood of the needle puncturing one of the many blood vessels surrounding the stomach, the pancreas, etc.

An example of the use of apparatus 100 with grasper 120 (or grasper 120a), as shown in FIGS. 6A-6F, may be to perform endoscopic sleeve gastroplasty. For example, shaft 104 of apparatus 100 may be endoscopically inserted into a patient's stomach after an initial marking is made on the anterior and posterior walls of the stomach, along the greater curve, to provide a guideline for the suturing procedure. The apparatus 100 may then deploy a first anchor at a distal location along the stomach wall where the sleeve is to begin, e.g. on a distal anterior or posterior wall. Optionally, apparatus 100 may begin the suturing from the distal location of the stomach, e.g. near the Pyloric part, and from there proceed in the proximal direction. Additional anchors may be attached to the anterior wall, the greater curve wall and the posterior wall of the stomach to create a desired suturing pattern, e.g. a triangle suture configuration. The suturing pattern may be performed with 2-6 anchor attachments, for example, to create an anchoring group. After the 2-6 attachments are made, the suture may be tightened and tied (or otherwise secured, without tying). Alternatively, the suture may be tightened only after additional attachments along the greater curve are made. A similar pattern may be performed with several more anchor attachments towards the proximal end of the stomach along the guideline, e.g. 4-10 anchoring units, to maximize the reduction of stomach size.

Another use example of apparatus 100 is to perform bariatric revision in a stomach after a sleeve gastroplasty. Due to expansion of the sleeve over time, a revision may be required. To perform the revision, a suturing line may be performed within the stomach along the previous sleeve gastroplasty suturing line, to further decrease the volume of the stomach. The new suturing line may extend along the stomach's new (post-sleeve gastroplasty) greater curve.

Another example of using apparatus 100 is for outlet revision. In some cases, after a gastric bypass is performed, the outlet expands, reducing the effect of the bypass. To reduce the expansion, the outlet circumference of the stomach bypass outlet to the bypass tunnel may be reduced. The apparatus 100 is used to suture the outlet. Anchors may be attached to the stomach walls at the outlet, and the suture may be tightened to narrow down the diameter of the outlet. This may be performed in multiple longitudinal locations along the expanded outlet.

Another example of using the apparatus may be to seal a perforation in the stomach, intestines, or any other luminal part of the gastric system. The procedure may be performed by attaching an anchor at an edge of the perforation. A second anchor may be attached to an opposite edge of the perforation. Additional anchors may be attached to create a crisscross suturing pattern across the perforation, e.g., two or four additional stitches. After attaching the anchors, the suture attached to the anchors is pulled to generate tension to seal the perforation.

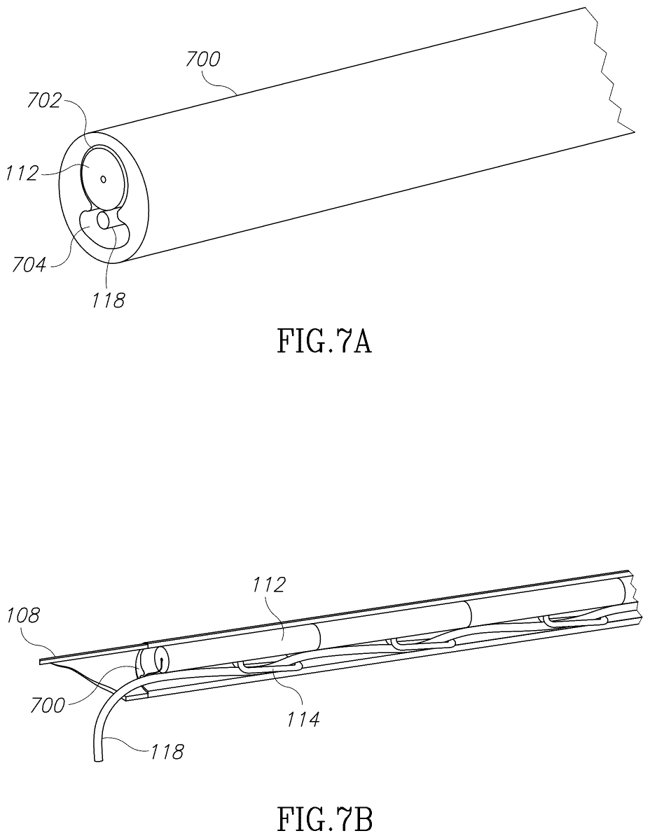

FIGS. 7A-7B show an inner guide shaft 700, which is optionally housed inside needle 108 and includes two inner channels terminating with two respective openings 702 and 704, for the separate advancement and deployment of the anchors and their extensions. The two channels may be longitudinally interconnected (to facilitate the interconnection between each anchor and its cord) but still allow for a series of anchors 112 to move along one channel, and a series of extensions 114 (and thread 118) to separately move along the other channel, separating these elements and preventing suture 118 from being entangled with anchors 112 and/or their extensions 114.

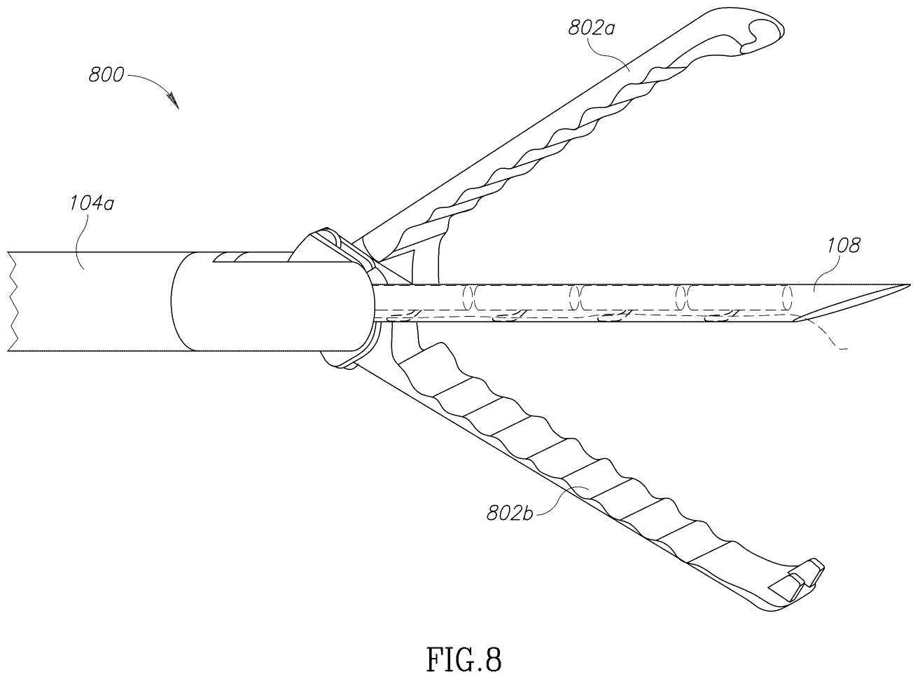

FIG. 8 shows a distal portion of an alternative apparatus 800 for suturing tissue, in which needle 108 is housed within a jawed grasper. This grasper may include two (or more) jaws 802a and 802b which are structured to allow needle 108 to reside between them when they are closed. The base of jaws 802a and 802b—the location where they connect to a shaft 104a—includes an opening of an elongated channel extending inside the shaft and housing needle 108. Needle 108 may be moved in the distal and proximal directions within that channel. For example, the distal portion of apparatus 800 may first be advanced towards the tissue to be sutured, while needle 108 is still fully housed within the channel in shaft 104a. Jaws 802a and 802b are then opened to encompass some of the tissue, and are closed over the tissue to grasp it. Then, needle 108 is pushed distally through the channel so that it penetrates the tissue. Needle 108 is advanced distally until its distal opening reaches beyond the tissue. The anchors (shown in phantom lines inside needle 108) are then pushed distally, until the foremost anchor is extracted from the needle to the space beyond the tissue. Finally,

needle 108 is retracted back into the channel in shaft 104a, jaws 802a and 802b are opened, and the distal portion of apparatus 800 may be relocated in order to deploy the next anchor at a different location of the tissue.

Optionally, shaft 104a of this grasper may be housed inside an external shaft, such as shaft 104 of the earlier figures. Alternatively, shaft 104a may be directly threaded through a work channel of an endoscope.

In an alternative configuration of any of the apparatuses for suturing tissue, they may be configured for partial-thickness suturing of soft tissue, instead of full-thickness suturing. In such configuration, the needle may penetrate only to a portion of the thickness of the tissue, and the anchors may be deployed inside the tissue (or partially inside the tissue) instead of beyond the tissue.

In addition, although the shaft, needle, grasper, and grasper protection sleeve are shown with a substantially concentric profile throughout most of the figures, one or more of them may be configured with an eccentric profile instead.

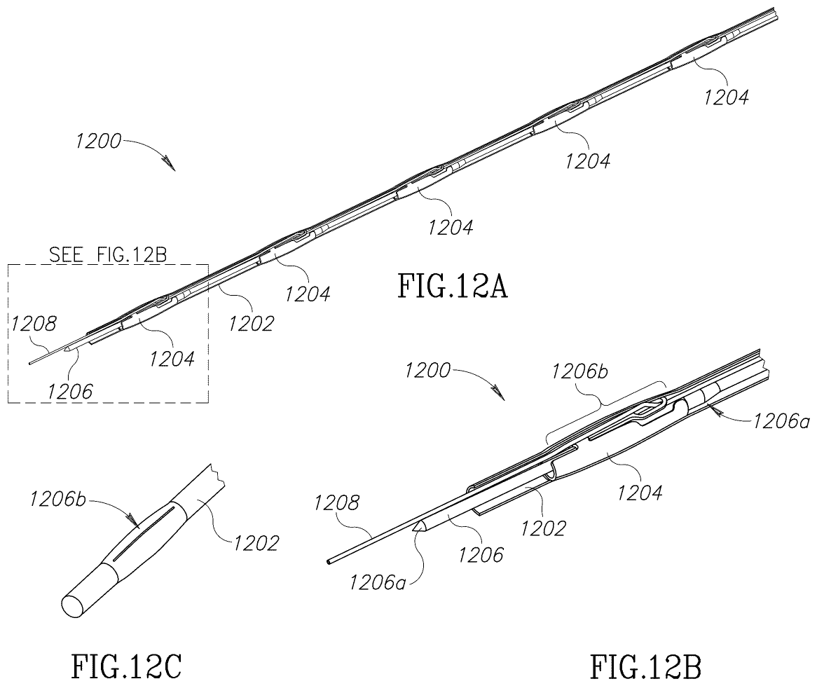

Reference is now made to FIG. 12A, which shows a cross-sectional view of a sleeve arrangement 1200 that may serve as an alternative to the shaft described above (referenced 104 or 104a in other figures), or may reside inside such shaft, replacing at least some of its internal components such as its anchors and suture. Reference is also made to FIG. 12B which is an enlargement of a distal portion of sleeve arrangement 1200 of FIG. 12A.

Advantageously, sleeve arrangement 1200 may include an internal ratchet mechanism which allows for unidirectional pushing of anchors, such as anchors 1204, in the distal direction. The ratchet mechanism may include a pushrod 1206 extending lengthwise along sleeve arrangement 1200, and having multiple bulges 1206a disposed (optionally equidistantly) along its length. Each such bulge 1206a may be connected to, attached to, or integrally formed with pushrod 1206, such that it is affixed to the pushrod as it moves. Each bulge 1206a may be configured with a generally conical shape, having the wider side of the cone pointing distally and the narrower side of the cone pointing proximally. Accordingly, when pushrod 1206 is pushed distally or pulled proximally, bulges 1206a move with it; as the pushrod is pushed distally, each bulge pushes its respective anchor 1204 with it, and as the pushrod is pulled proximally, each bulge passes through its respective anchor until it is positioned immediately behind it, ready for the next push operation. This will be further discussed below.

An alternative ratchet mechanism (not shown) may include a unidirectional extension on each anchor, such as a fin extending outwardly and proximally from the anchor, and a series of matching bulges on the sleeve or other tube which encircle the anchors. This way, the anchors can only move forward (distally) within that tube and not return backwards (proximally). Such unidirectional extension may also aid in preventing the anchor from returning back through the tissue aperture after it has been implanted beyond the tissue.

Sleeve arrangement 1200 may include a sleeve 1202 made of a flexible material, such as Polytetrafluoroethylene (PTFE, commercially known as Teflon®) or the like. Anchors 1204 may be positioned (optionally equidistantly) inside sleeve 1202, with a suture 1208 threaded through them and also disposed within the sleeve.

Sleeve 1202 may be configured with a structure-weakening alteration at each of its areas where anchors 1204 are to reside, so that the anchors become relatively secured at these areas when pushrod 1206 is immobile. These structure-weakening alteration may be, for example, elongated slits, such as slit 1206b shown in FIG. 12B but more clearly visible in FIG. 12C, to which interim reference is now made. FIG. 12C which shows a perspective view of a distal portion of sleeve 1202, corresponding to its distal portion as shown in FIGS. 12A and 12B. As shown, sleeve 1202 includes a slit 1206b along an area where the distalmost anchor (not seen in this figure) is disposed. Each of slits 1206b may have a length that equals ±25% the length of each of the anchors. Slits 1206b weaken the structure of sleeve 1202 and allow its diameter to slightly expand when an anchor is disposed in the area of a slit.

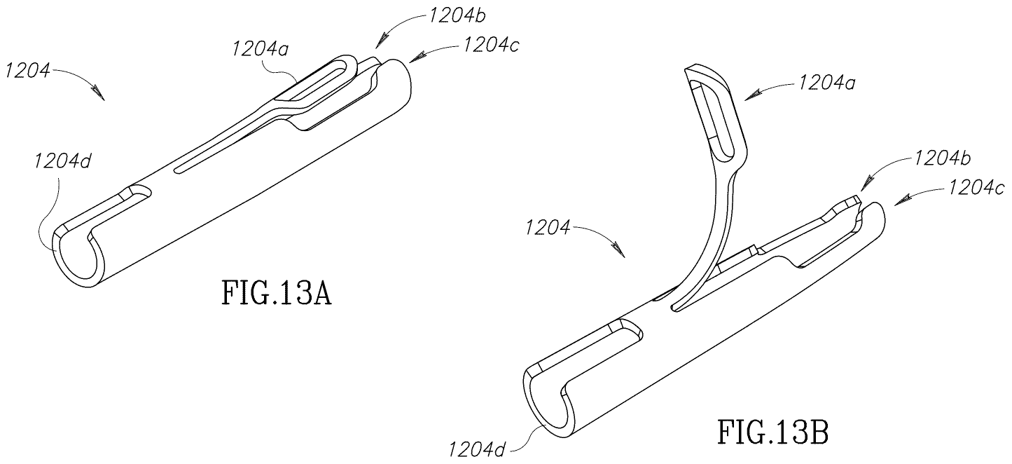

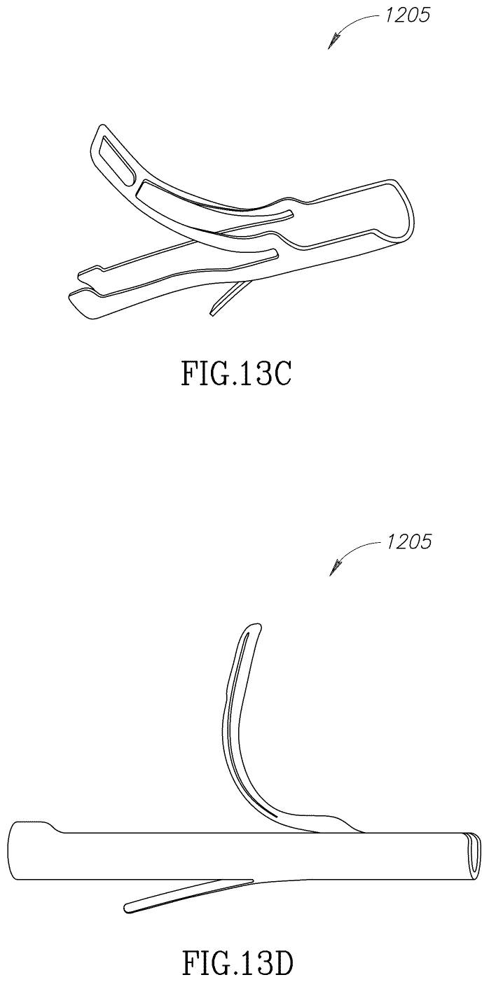

Reference is now made to FIGS. 13A and 13B which show anchor 1204 in greater detail. In FIG. 13A, anchor 1204 is shown with an extension 1204a lying flush with the anchor's body, while in FIG. 13B, the anchor is shown with that extension emerging perpendicularly from the body.

Anchor 1204 may be generally similar to anchor 112d described above (with reference to FIGS. 3F-3H), except for some differences. Anchor 1204 may have an elongated body and a flexible extension 1204a emerging from it, as discussed above for anchor 112d. However, anchor 1204 may include a feature in its proximal side which enables it to be unidirectionally pushed by the ratchet mechanism of sleeve arrangement 1200, described above: the circumference of the anchor's hollow body, at its proximal side, may not be a complete circle but rather a pair (or more) of concave and resilient ‘legs’ 1204b-c pointing towards one another, and defining an internal diameter therebetween (at their proximal ends) which is slightly smaller (e.g., by 5-30%) than the internal diameter of the rest of the anchor's body. This smaller internal diameter is maintained as long as legs 1204b-c are at a resting state, not biased by any force. With reference also to FIG. 12B, bulge 1206a may have its larger diameter (at its distal side) be similar (or slightly smaller, e.g., by 1-10%) of the internal diameter of anchor 1204 at its non-legged area, but therefore slightly larger than the internal diameter of legs 1204b-c at their resting state. Therefore, when pushrod 1206 is pushed distally, a distal edge of bulge 1206a engages the proximal edges of legs 1204b-c, causing the entire anchor 1204 to move distally. Conversely, when pushrod 1206 is pulled proximally back through anchor 1204, the smaller diameter at the bulge's 1206a proximal side may slide through the non-legged area of anchor 1204, and, as it reaches legs 1204b-c, expand and bias them outwardly until the bulge completely passes them and positions itself immediately behind the anchor (namely, immediately behind the proximal ends of the legs). Then, when the outward biasing of legs 1204b-c ceases, they return to their resting state, ready for another push operation.

Optionally, legs 1204b-c are configured to slightly expand outwardly (such as by thermal training) when not biased by any force. This may assist in preventing anchor 1204 from sliding back through the tissue aperture when the pushrod on which it is mounted (to be discussed below) is pulled back from the tissue, by expanding the profile of the anchor at its proximal end to be much larger than the tissue aperture.

Legs 1204b-c may also be configured with enough space therebetween to allow the thread (on which the series of anchors is threaded) to conveniently pass in that space.

Reference is now made to FIGS. 13C-13D, which show another configuration of an anchor 1205 in a perspective view and a side view, respectively. Anchor 1205 may be similar to anchor 1204 expect for the following differences: First, anchor 1205 may have a fin at its lower side, as discussed above. Second, the entire upper wall of anchor 1205 may be open, except for the loop at the end of the flexible extension; this may allow the thread to pass more freely over the anchor when the anchor is within its encapsulating sleeve or other tube.

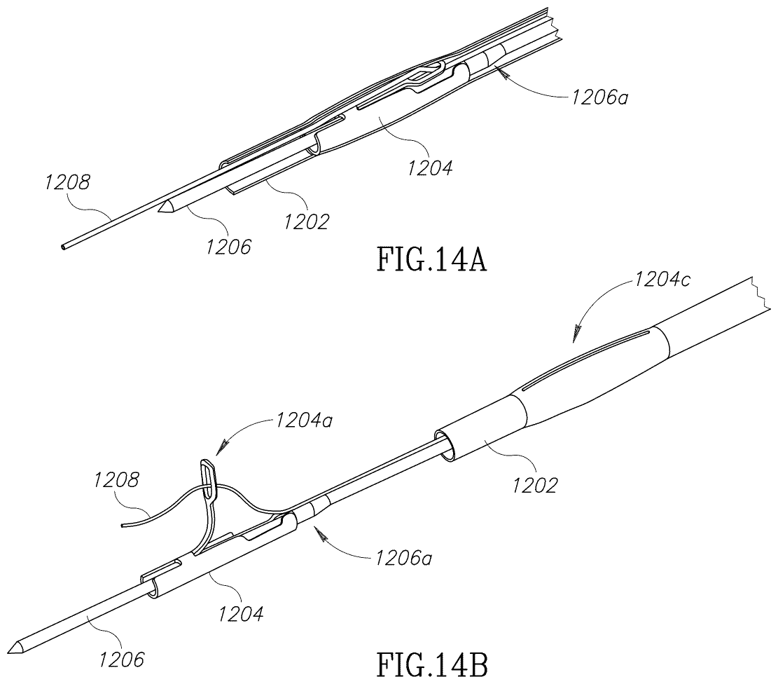

FIGS. 14A and 14B illustrate a typical push operation which takes place using the ratchet mechanism of sleeve arrangement 1200. FIG. 14A shows the distalmost anchor 1204 in its designated area within sleeve 1202, prior to pushing. Then, pushrod 1206 is pushed distally, and bulge 1206a engages anchor 1204 and causes it to also travel distally. As anchor 1204 emerges out of sleeve 1202 (and optionally also out of any other encapsulation not shown here, such as a shaft, a needle, or a channel of an endoscope), its extension 1204a may become unbiased and erect. Next, pushrod 1206 may be pulled proximally, so that bulge 1206a passes through the anchor next in line inside sleeve 1202; this anchor is hidden in FIG. 14B, and is located inside a slightly widened (weakened) area 1202c of the sleeve. Then, as pushrod 1206 is positioned immediately behind that anchor, another push operation may take place, and so on and so forth until all the anchors inside sleeve arrangement 1200 are deployed.

Notably, FIGS. 12A-14C do not illustrate any grasping means nor a needle, as these elements may be the same as described above with reference to other embodiments. For example, sleeve arrangement 1200 may be disposed inside such needle and/or grasper, which serve to pierce and grasp the tissue as discussed above. Alternatively, the embodiment shown in FIGS. 12A-14C may not necessitate a needle that encircles the anchors, and may instead rely on an optional sharp end 1206a of pushrod 1206 to penetrate the tissue, and optionally also on a distal rim 1204d of anchor 1204 being optionally sharp and chamfered, and thus capable of further expanding an initial piercing performed by that sharp end of the pushrod. The sharp end 1206a of pushrod 1206 may be a solid, sharpened spike, as shown (which pricks the tissue and then widens the initial aperture as it pushed further into the tissue), or a hypodermic needle (which actually cuts the tissue to approximately the shape of the needle's outer diameter).

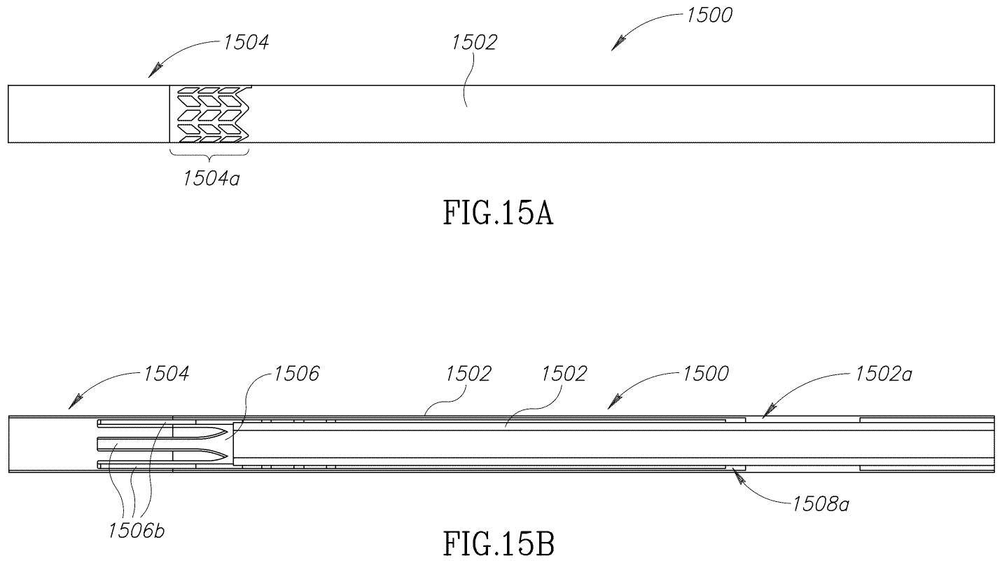

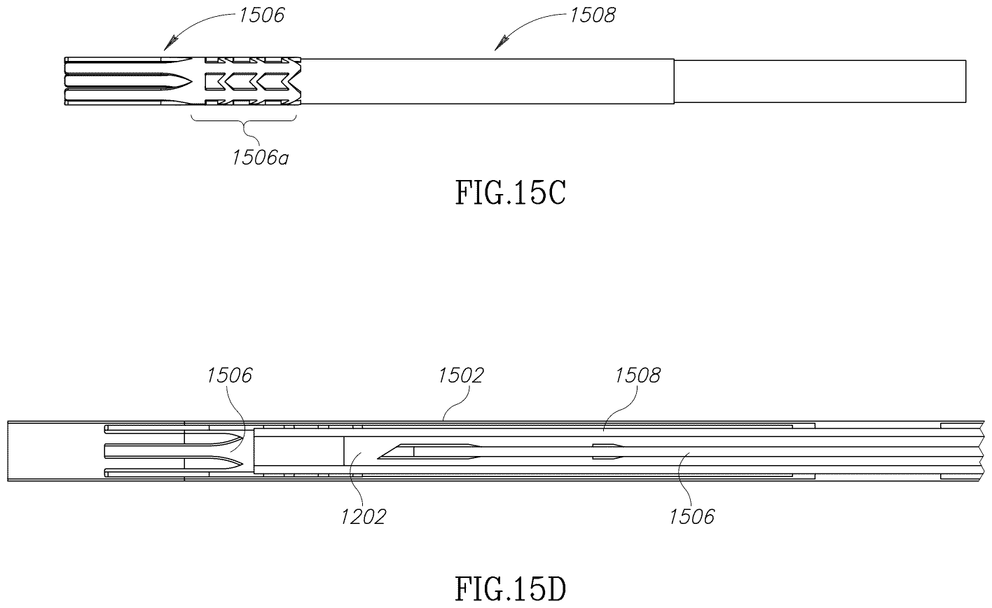

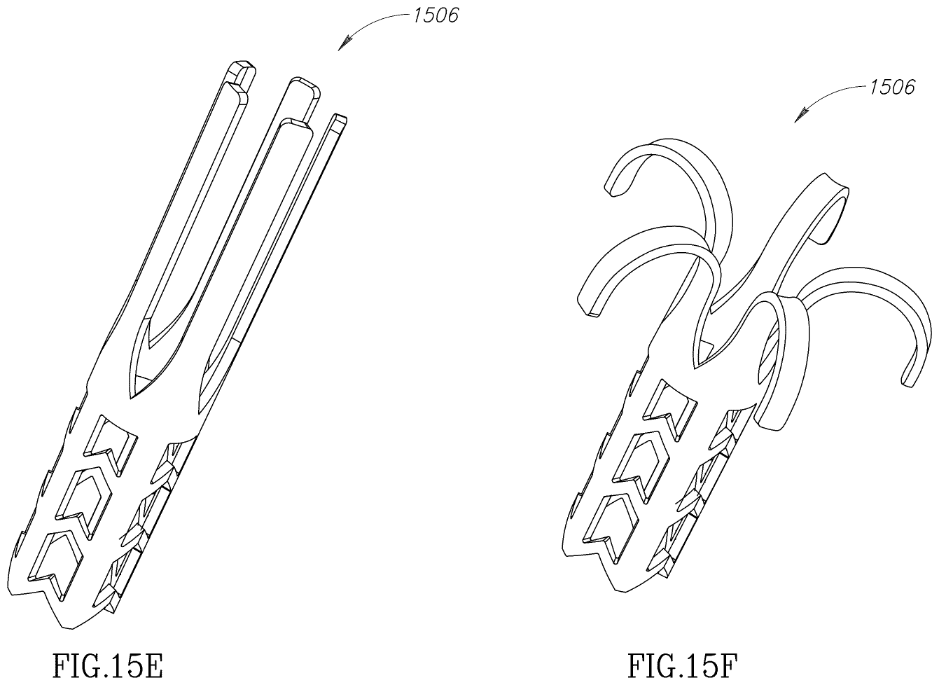

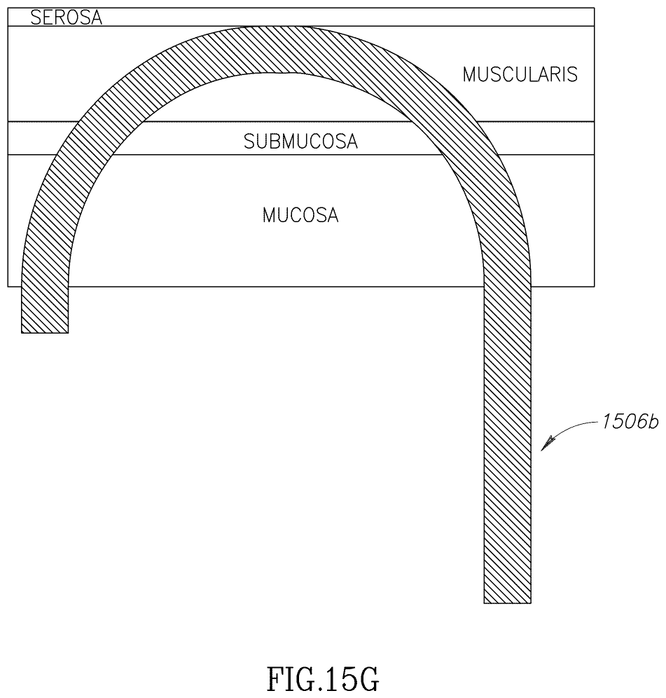

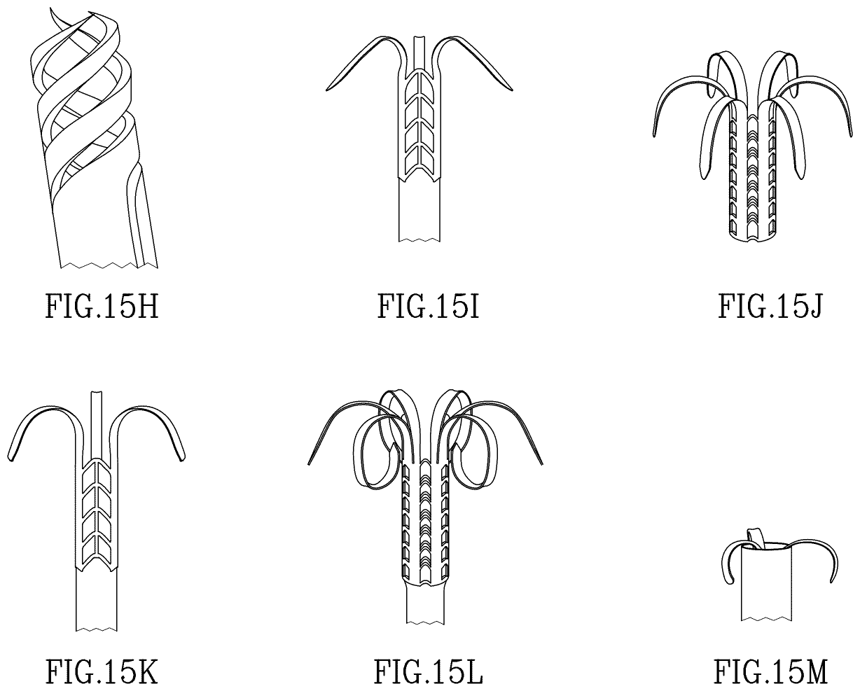

Reference is now made to FIGS. 15A-15C which show a shaft arrangement 1500 for use with sleeve arrangement 1200 of FIGS. 12A-14C and its internal components. Shaft arrangement 1500 may be used instead of the shaft referenced 104 or 104a in earlier figures. FIG. 15A is an external view of an outer shaft 1502 of shaft arrangement 1500, FIG. 15B is a cross-sectional view of the shaft arrangement, and FIG. 15C is an external view of the inner tube.

Shaft arrangement 1500 may include outer shaft 1502 that is similar to shaft 104 in the sense that it may be a flexible elongated tube, made, for example, of plastic and/or braided metal, and configured to be flexibly manipulated inside an endoscope. Shaft arrangement 1500 may further include an inner tube 1508, discussed further below.

Outer shaft 1502 may have a length of, for example, 200-3000 mm, wherein part of this length, such as between 10-300 mm, may be disposed inside a handle, which may be similar to handle 102 discussed with reference to earlier figures. Outer shaft 1502 may have an external diameter of, for example, 2 to 10 mm. Outer shaft 1502 may have a uniform or a variable diameter along its length.

A distal tube 1504 may be disposed at the distal end of outer shaft 1502, and affixed to the outer shaft by an adhesive or other fixation means. Distal tube 1502 may be made of a rigid material, such as stainless still or rigid plastic. The internal diameter of distal tube 1504 may be slightly larger than the external diameter of the distal area of outer shaft 1502, such that distal tube may encircle the distal area of the outer shaft where they are affixed to one another. Distal tube 1504, at its proximal side, may have a perforated wall 1504a such that, if an adhesive is used for the fixation, the adhesive not only contacts the outer wall of outer shaft 1052 and the inner wall of the distal tube, but also fills the perforations and provides additional resistance to separation of the distal tube and the outer shaft.

Distal tube 1504 may be aimed at encircling a grasping means, which may be configured like the grasping means discussed with reference to earlier figures, or as shown in FIGS. 15E-15F. discussed further below.

FIG. 15D shows a cross-sectional view of sleeve arrangement 1200 of FIG. 12A with shaft arrangement 1500 of FIGS. 15A-15C, but without the anchors mounted on pushrod 1206 (for better clarity).

An exemplary grasper 1506, shown in FIGS. 15E-15F, may be made of the same material of the earlier-described grasping means, and include a tubular base 1506a and multiple concentric tines 1506b (such as 3-8 tines, or 5 tines as shown) extending distally from the base. Tines 1506b may be biased when resting in and delimited by distal tube 1504 of FIG. 15A, such that grasper 1506 obtains on overall tubular shape consisting of the tines and the base, as shown in FIG. 15E. When tines 1506b exit distal tube 1504 distally and are no longer biased by it, they assume their expanded, unbiased position which is shown in FIG. 15F.

The mechanism of action of grasper 1506 may be similar to that of some of the earlier-described grasping means: as grasper 1506 is pushed outside of distal tube 1504, the tines begin penetrating the tissue in front of the distal tube, and travel mostly sideways inside the tissue as they continue expanding outwardly. This way, ideally, the tines do not even exit the tissue to its distal side, but remain wholly inside the tissue as they expand, and optionally even exit the tissue back to its proximal side, as they curl backwards and assume a hook shape. This helps prevent damage to other tissues (organs, blood vessels, etc.) that are located on the other side of the tissue. For example, in a surgical procedure performed in the stomach, illustrated in FIG. 15G (which is not drawn to scale), a tine 1506b may fully penetrate the three inner layers of the stomach wall (mucosa, submucosa, and muscularis), and only partially penetrate (or not penetrate at all) the outermost layer of the stomach-the serosa. It is also possible for the tines to not even reach the serosa, but rather penetrate fully through the mucosa and submucosa, and penetrate partially through the muscularis externa. Even in case one or more of the tines happens to fully penetrate the tissue (namely, beyond the serosa), this may likely happen only when these tines are already curled sideways or even backwards, so damage to other nearby tissues (for example, nearby organs, blood vessels, etc.) is unlikely.

Each of tines 1506b may have a wall thickness of 0.15-0.30 mm (for example, 0.26 mm), a width of 0.50-1.10 mm (for example, 0.8 mm), and a length from its distal end to where base 1506a begins) of 5-15 mm (for example, 7.40 mm). Notably, the distal ends of each tine 1506b may be blunt, and shaped as a triangle with slightly round edges (a diameter of, e.g., 0.10-0.30 mm of the round edges). Each of tines 1506b may gradually expand in width as it approaches base 1506a, to strengthen the tine in that transition area. Alternatively, each of the tines may be rectangular along its entire length (now shown). The overall diameter of base 1506a and tines 1506a, when they are collapsed, may be 2.0-3.0 mm (for example, 2.48 mm), and the overall diameter of the tines, when they are fully expanded to their hook shape, may be 2.50-3.50 mm (such as 3.00 mm).

The wall thickness of tines 1506b may be selected such that their distal ends are not too sharp, and therefore will not damage other tissue that is located behind the grasped tissue even if one or more of the tines incidentally does emerge on the other side of the grasped tissue. The configuration of grasper 1506 (its shape, size, thickness, superelasticity, elasticity, resiliency, thermal training, etc.) may be such that a force of between 500-1500 grams (or, more specifically, 700-1100 grams) is required in order to pull back and collapse the grasper back into distal tube 1504. This implies that the force at which grasper 1506 expands upon its extraction from distal tube 1504 is quite significant, so that tines 1506b will remain secured to the tissue at their hook shape throughout the anchor implantation procedure (further discussed below).

Further in FIGS. 15B-15C there is shown inner tube 1508 which is affixed to grasper 1506. Inner tube 1508 is disposed inside outer shaft 1502 of FIG. 15A. Sleeve arrangement 1200 of FIG. 12A, in turn, is disposed inside inner tube 1508. Inner tube 1508 may be made of a flexible material, configured to be flexibly manipulated inside an endoscope.

The internal diameter of base 1506a of grasper 1506 may be slightly larger than the external diameter of the distal area of inner tube 1508, such that base may encircle the distal area of the inner tube where they are affixed to one another. Base 1506a, at least at its proximal side, may have a perforated wall, such that, if an adhesive is used for the fixation, the adhesive not only contacts the outer wall of inner tube 1508 and the inner wall of the base, but also fills the perforations and provides additional resistance to separation of grasper 1506 and the inner tube.

In order to extract tines 1506b of grasper 1506 out of distal tube 1504, inner tube 1508 may be pushed from the handle (not shown in this figure) relative to distal tube 1504.

Reference is now made to FIGS. 15H-15M, which show six alternative configurations of a grasper, which are different from grasper 1506 by the number and shape of tines. The configurations of FIGS. 15H-15K may be suitable for penetrating the full thickness of the tissue, while the configuration of FIGS. 15L-15M may be suitable for penetrating only the sub-serosa layers and optionally also some of the thickness of the serosa. The configuration of FIG. 15L is unique in that it provides a two-step grasping of the tissue: first, all tines penetrate the tissue; as the grasper continues to be pushed toward the tissue, the longer tines curl back and exit the tissue proximally, each curling to almost 360 degrees (or even more) over itself, enhance the securing of the tissue.

Outer shaft 1502 and inner tube 1508 may have complementary structures that prevent tines 1506b from being pulled too far distally where they can damage and/or become entangled with sleeve arrangement 1200, particularly with the flexible and delicate sleeve 1202. These complementary structures may be a step 1502a on the inner wall of outer shaft 1502 and an opposite step 1508a on the outer wall of inner tube 1508, such that the outer shaft cannot be pulled too much proximally. Alternative complementary structures may also be possible (but not shown), such as inversely-shaped steps or any other structure that physically prevents relative motion of the outer shaft and inner tube beyond a certain range.

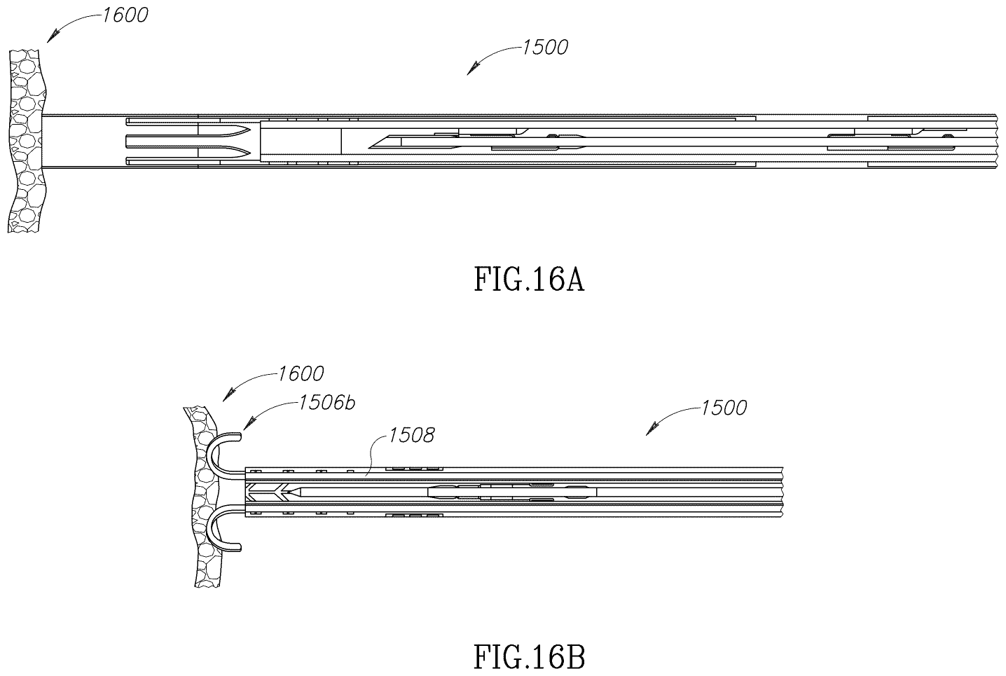

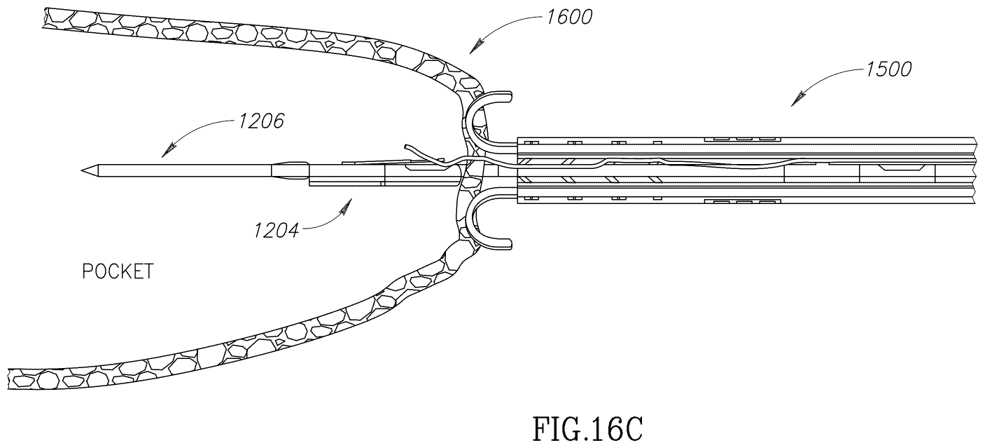

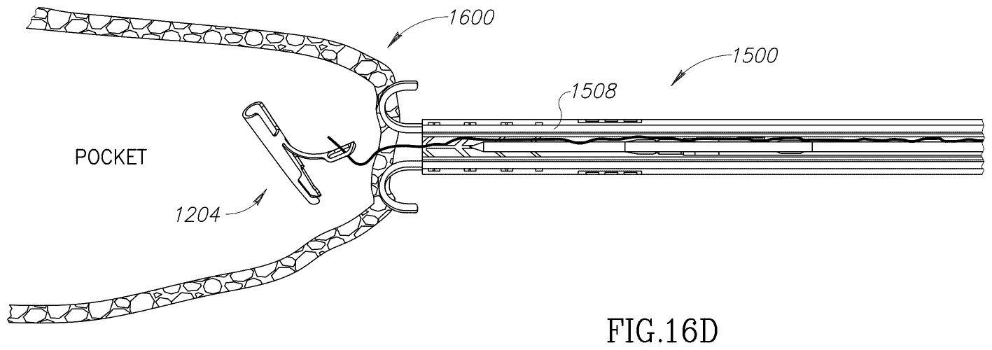

The overall interaction of shaft arrangement 1500, sleeve arrangement 1200, inner tube 1508, and all their related components may be better understood by referring to FIGS. 16A-16D. These figures illustrate four stages of implanting anchor 1204 beyond tissue 1600.

In FIG. 16A, the entire shaft arrangement 1500, with all its internal components, is brought adjacent to or in contact with the inner wall of the tissue 1600. Shaft arrangement 1500 may be disposed, for example, inside a work channel of an endoscope (not shown), such as a gastroscope or a colonoscope, with only a distal portion of the shaft arrangement (e.g., its distalmost 5-30 mm) exposed beyond the endoscope's distal end.

In FIG. 16B, inner tube 1508 is pushed distally, so that tines 1506b penetrate the tissue 1600 (ideally, not to its full thickness) and grasps it securely.

Then, in FIG. 16C, inner tube 1508 (together with outer shaft 1502) is pulled proximally, pulling the grasped tissue 1600 with it, while maintaining sleeve arrangement 1200 in place. This, simultaneously, creates a sort of pocket in the tissue 1600, and causes the sharp end of pushrod 1602 to penetrate the full thickness of the tissue and into that pocket. The distalmost anchor 1204 moves together with pushrod 1602, and also passes to the other side of the tissue 1600.

Next, in FIG. 16D, pushrod 1602 is pulled proximally back into inner tube 1508, and passes through anchor 1204, while the proximal end of the anchor is stopped by the rim of the aperture in the tissue and is prevented from moving back through the tissue. Anchor 1204 is thus released from pushrod 1602 and remains at the distal side of the tissue 1600, inside the pocket. Optionally, base 1506a of grasper 1506 is configured with a unidirectional bulge (not shown) on its inner surface, so that it aids in peeling anchor 1204 off pushrod 1602 as the pushrod is pulled back through the tissue aperture. This further helps prevent anchor 1204 from remaining on pushrod 1602 as the pushrod is pulled back.

Then, grasper 1506 may be released from the tissue 1600 by pulling it back into distal tube 1504 and preparing for the next anchor implantation sequence.

The technique illustrated in FIGS. 16A-16D and described above, in which a tissue pocket is created, the tissue is punctured, and an anchor is implanted on the other side of the tissue, may also be facilitated by other embodiments of the apparatus, grasper, and anchor, by operating them according to a similar principle.

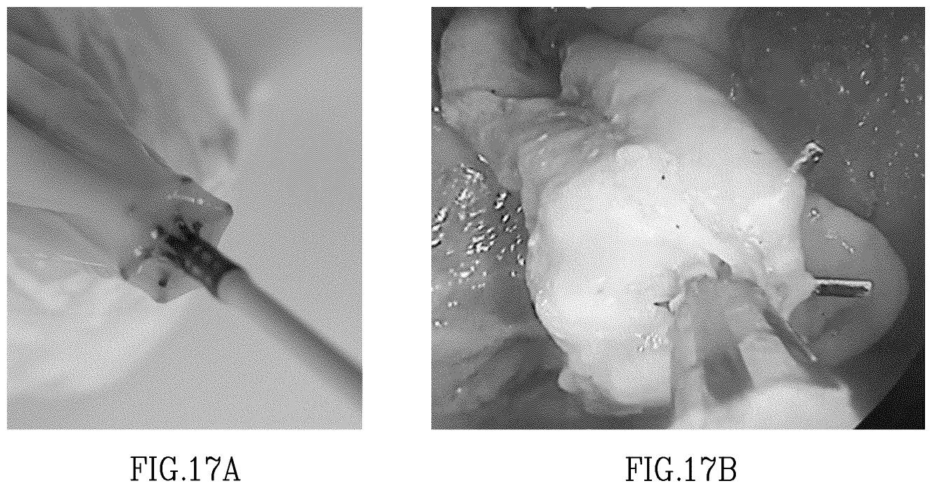

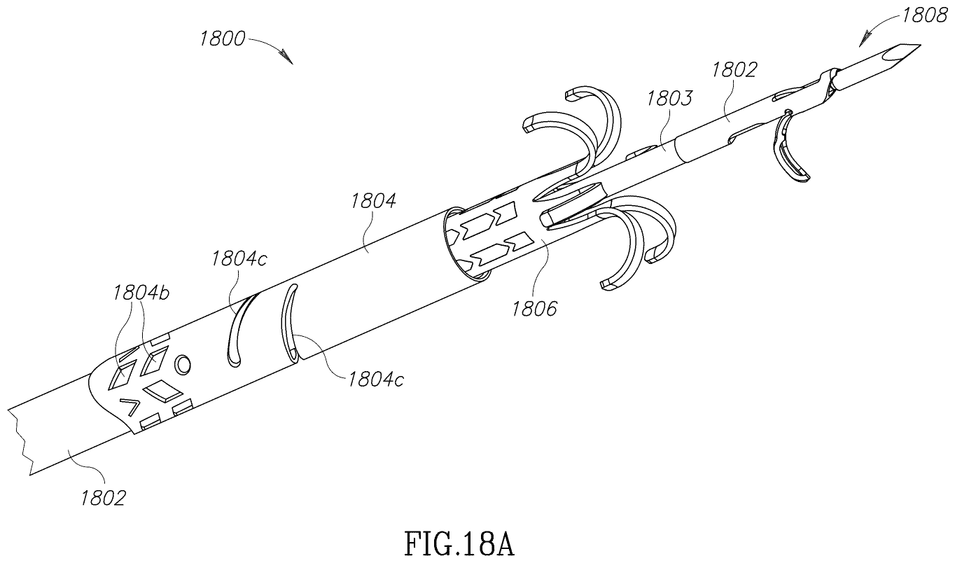

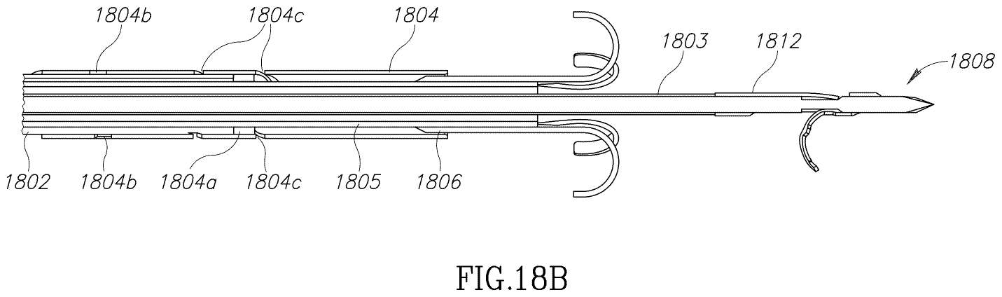

Reference is now made to FIG. 17A, which is a photograph of a porcine experiment performed with a version of grasper 1506 having six tines instead of five (such as a grasper 1806 shown in FIGS. 18A-18B). The photograph was taken after the grasper's tines have been fully expanded and the grasper has been pulled proximally to create a tissue sleeve/pocket, and nicely shows the tines threaded through the stomach wall but not penetrating fully through the serosa.

Additional reference is made to FIG. 17B, which is a photograph taken in another porcine experiment, this time with a five-tined grasper (such as grasper 1506 of FIGS. 15E-15F). The photograph shows the sleeve/pocket created by the stomach wall as the grasper was pulled proximally, as well as that two of the grasper's tines have in fact fully penetrated the stomach wall and are seen protruding laterally; this demonstrates that even in the event that one or more of the tines happen to penetrate the full thickness of the tissue, their lateral orientation when doing so (as well as their potential further curling backwards) is likely to prevent damage to organs at the other side of the penetrated tissue.

Reference is now made to FIGS. 18A-18B, which show a perspective view and a cross-sectional view, respectively, of a distal area of a shaft arrangement 1800 that may serve as an alternative to shaft 104/104a, shaft arrangement 1500, and/or sleeve arrangement 1200 discussed above. More specifically, elements of FIGS. 18A-18B may be generally similar to those shown in FIGS. 12A-15G and 16A-16D and discussed above, with the main difference being that the embodiment of FIGS. 18A-18B does not include a flexible sleeve 1202 which houses a series of anchors; instead, it may be configured to house a single anchor at any given time. Besides that, elements of FIGS. 18A-18B are intended to be similar to elements of the earlier figures if they bear the same name, with the exception of specific differences made apparent by the following discussions; the same applies to the functionality (method of operation) of such corresponding elements.

From the inside out, shaft arrangement 1800 may include some or all of the following: a needle 1808; an anchor 1812 configured to be mounted over the needle; an anchor backer 1803 mounted over the needle, behind the anchor; an inner tube 1805 mounted over the anchor backer; a grasper 1806 attached to, connected to, or integrally formed with the inner tube; an outer shaft 1802 mounted over the inner tube; a distal tube (also “grasper collector”) 1804 whose proximal area is fixedly mounted over the outer shaft, and whose distal area is configured to cover the grasper and the inner tube; and a stopper ring 1804a attached to, connected to, or integrally formed with the inner wall of the grasper collector, adjacent the distal end of the inner tube (for example, if the stopper ring and the grasper collector are both metallic, they may be welded together).

Similar to what is shown in FIGS. 16A-16D, shaft arrangement 1800 may be used to implant multiple anchors beyond a tissue, with a suture (not shown in FIGS. 18A-18B to prevent obstruction of view) that interconnects the anchors similarly to the above descriptions. In operation, a length of approximately 6 to 10 mm of the distal area of shaft arrangement 1800 may be exposed beyond the distal end of the endoscope (namely, beyond the distal opening of the endoscope's work channel) before actuation of the various parts of the shaft arrangement; at this stage, these various parts may still be covered by outer shaft 1802 and/or grasper collector 1803 so that they cannot damage any tissue.

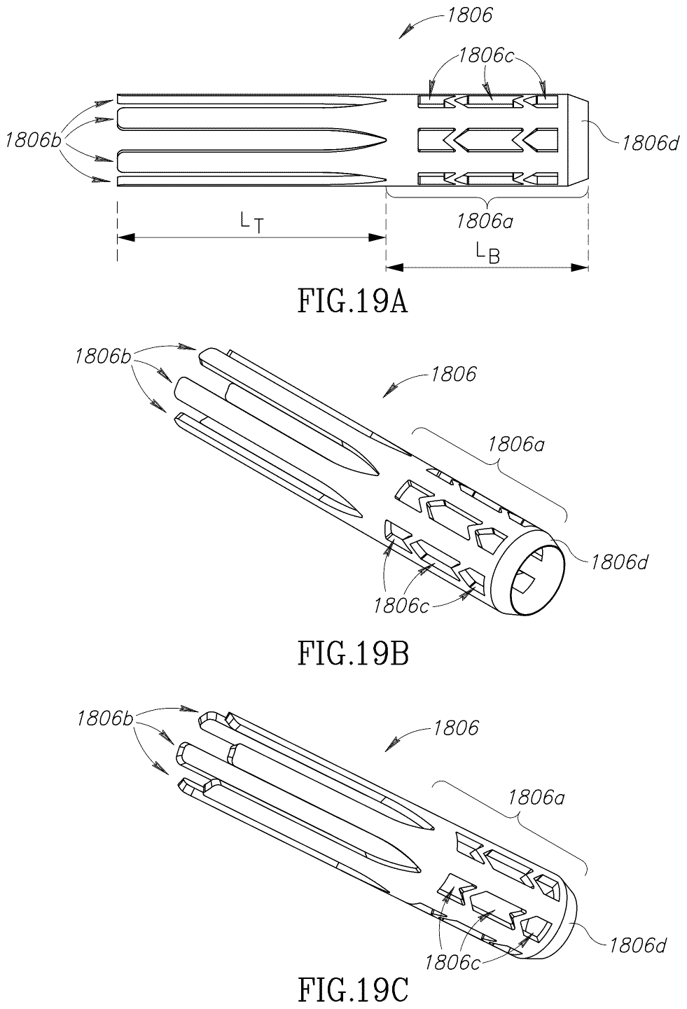

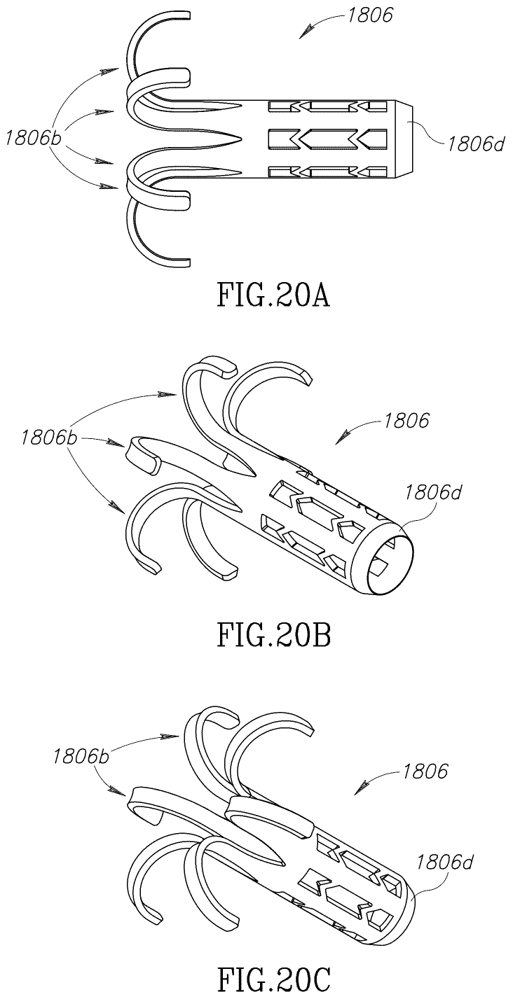

FIGS. 19A-19C and 20A-20C show grasper 1806 in greater detail, in a closed (biased) configuration and an open (expanded) configuration, respectively. Grasper 1806 may be similar to grasper 1506 of FIGS. 15E-15F, and include a tubular base 1806a and multiple concentric tines 1806b, for example six tines as shown (but optionally anywhere between 3 to 8 tines).

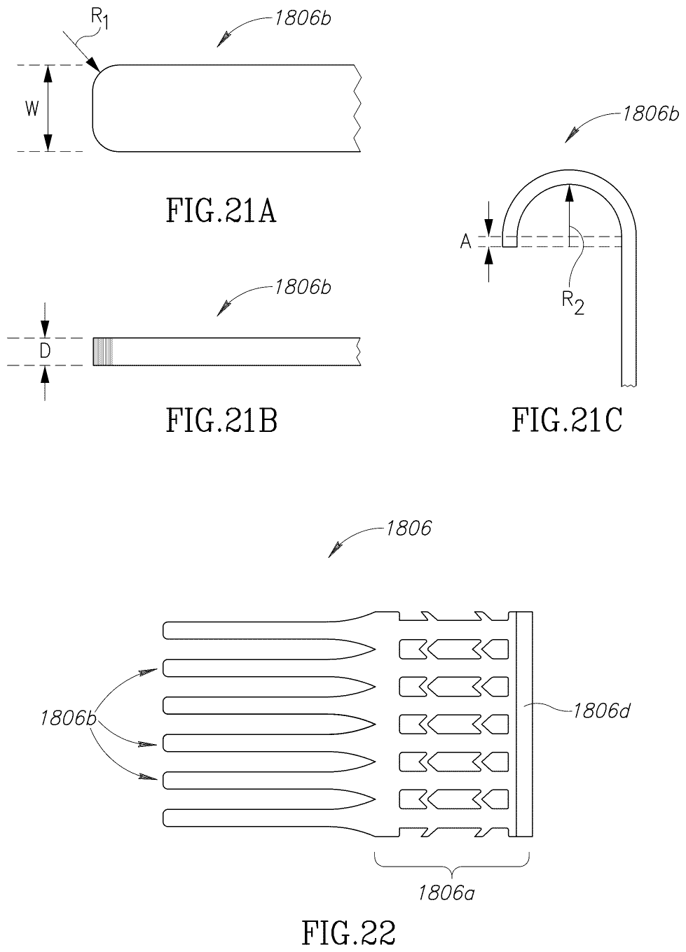

Grasper 1806 may be fabricated from a planar sheet of material, as shown in FIG. 22, for example by laser cutting. The sheet may be rolled to form the tubular shape of grasper 1806. The sheet may be secured in its tubular shaped using, for example, an adhesive between tubular base 1806a and inner tube 1805. A series of optional apertures 1806c in tubular base 1806a may reinforce the interconnection between the tubular base and inner tube 1805, by allowing some of the adhesive to enter the apertures and dry or cure in them, thereby further preventing grasper 1806 from detaching and freely rolling over the inner tube. In the example of FIGS. 19A-19C and 20A-20C, grasper 1806 includes 18 such apertures 1806c, arranged in 6 concentric groups each including 3 apertures arranged longitudinally. Apertures 1806c may occupy an area equivalent to between 10-70% of the total circumferential area of tubular base 1806a, or more specifically 10-30%, 20-40%, 30-50%, 40-60%, or 50-70%.

Grasper 1806 may proximally end with a chamfer 1806d, to smoothen the grasper's collection into grasper collector 1804 (or outer shaft 1802, in the absence of a grasper collector) by preventing or minimizing collision of the proximal end of the grasper with the distal end of the grasper collector (or the outer shaft).

Grasper 1806 optionally has the following measurements, which advantageously enable it to fit within a relatively narrow work channel of an endoscope, but still fulfill its tissue grasping function successfully: The overall length (L_{T}+L_{B}) of grasper 1806 may be 10 to 20 mm (or alternatively beyond that), for example 10-13 mm, 12-16 mm, 14-17 mm, 16-19 mm, or 18-20 mm. The length of tubular base 1806a (L_{B}) may be 3-8 mm (or alternatively beyond that), for example, 3-5 mm, 4-6 mm, 5-7 mm, or 6-8 mm. The length of tines 1806b (L_{T}) may be 5-13 mm (or alternatively beyond that), for example, 5-7 mm, 7-9 mm, 9-11 mm, or 10-13 mm. Grasper 1806 may have an outer diameter of approximately 1.5-4.5 mm (or alternatively beyond that), for example, 1.5-2.5 mm, 2-3 mm, 2.5-3.5 mm, 3-4 mm, or 3.5-4.5 mm.

Inner tube 1805 may be made, for example, of PEEK, stainless steel, or a similar material.

With reference to FIG. 21A, which is a top view of a distal area of one of tines 1806b, each tine may have a width (W), at least at its distal end, of 0.3 to 1.5 mm (or alternatively beyond that), such as 0.3-0.5 mm, 0.4-0.6 mm, 0.5-0.7 mm, 0.6-0.8 mm, 0.7-0.9 mm, 0.8-1.0 mm, 0.9-1.1 mm, 1.0-1.2 mm, 1.1-1.3 mm, 1.2-1.4 mm, or 1.3-1.5 mm. The distal end of the tine may be perpendicular to the longitudinal axis of grasper 1806, and may be blunt, namely—it may have rounded corners to delicately facilitate tissue penetration and reduce the likelihood that the tines fully penetrate to the other side of the tissue. The radius (R₁) of each rounded corner may be 0.05 to 0.75 mm (or alternatively beyond that), such as 0.05-0.20 mm, 0.15-0.30 mm, 0.25-0.40 mm, 0.35-0.50 mm, 0.45-0.60 mm, 0.55-0.70 mm, or 0.65-0.75 mm. Each of tines 1806b may have a uniform width along the majority of its length, such as approximately the distal 80% of its length, as seen in FIGS. 19A-19C. Alternatively, the tines may be gradually narrowing towards their distal ends, each having an elongated trapezoid shape.

With reference to FIG. 21B, which is a side view of the same distal area of one of tines 1806b, each tine may have a thickness (D), at least at its distal end, of 0.1 to 0.85 mm (or alternatively beyond that), such as 0.10-0.25 mm, 0.20-0.35 mm, 0.30-0.45 mm, 0.40-0.55 mm, 0.50-0.65 mm, 0.60-0.75 mm, or 0.70-0.85 mm. This may also be the thickness of the sheet of material of which grasper 1806 is made, as shown in FIG. 22.

Tines 1806a (or relevant portions thereof) may be trained (for example, using heat treatment as known in the art) so that they assume the shape shown in FIGS. 20A-20C when not biased by grasper collector 1804 (or by an outer tube, if a grasper collector is not used). With reference to FIG. 21C, which is a side (profile) view of one of tines 1806b in its expanded (unbiased) state, each tine may curl backwards at an angle of approximately 180 degrees as shown, or more generally between 145 and 205 degrees, such as 145-160 degrees, 155-170 degrees, 165-180 degrees, 175-190 degrees, 185-200 degrees, or 195-205 degrees. The internal radius of curvature (R₂) of the curled area of the tine may be 1 to 5 mm (or alternatively beyond that), such as 1.0-2.0 mm, 1.5-2.5 mm, 2.0-3.0 mm, 2.5-3.5 mm, 3.0-4.0 mm, 3.5-4.5 mm, or 4.0-5.0 mm. Optionally, the tine may have a straight (not curled) distalmost area (A), distal to the curled area, having a length of approximately 0.5 to 5.0 mm (or alternatively beyond that), such as 0.5-2.0 mm, 1.5-3.0 mm, 2.5-4.0 mm, or 3.5-5.0 mm.

With reference back to FIGS. 19A-19C and 20A-20C, chamfer 1806d may have an angle of 10-45 degrees (or beyond that) relative to the longitudinal axis of grasper 1806, such 10-25 degrees, 20-35 degrees, or 30-45 degrees. Chamfer 1806d may occupy a length of 0.3-1.4 mm (or alternatively beyond that) out of the length (L_{B}) of tubular base 1806a, such as 0.3-0.6 mm, 0.5-0.8 mm, 0.7-1.0 mm, 0.9-1.2 mm, or 1.1-1.4 mm.

When expanded (unbiased), grasper 1806 may be configured to apply a grasping force of approximately 0.5 to 2.0 kilograms (kg) to the tissue, such as 0.5-0.8 kg, 0.7-1.0 kg, 0.9-1.2 kg, 1.1-1.4 kg, 1.3-1.6 kg, 1.5-1.8 kg, or 1.7-2.0 kg. This grasping force capability may be the result of the material tines 1806a are made of, their training, and their measurements.

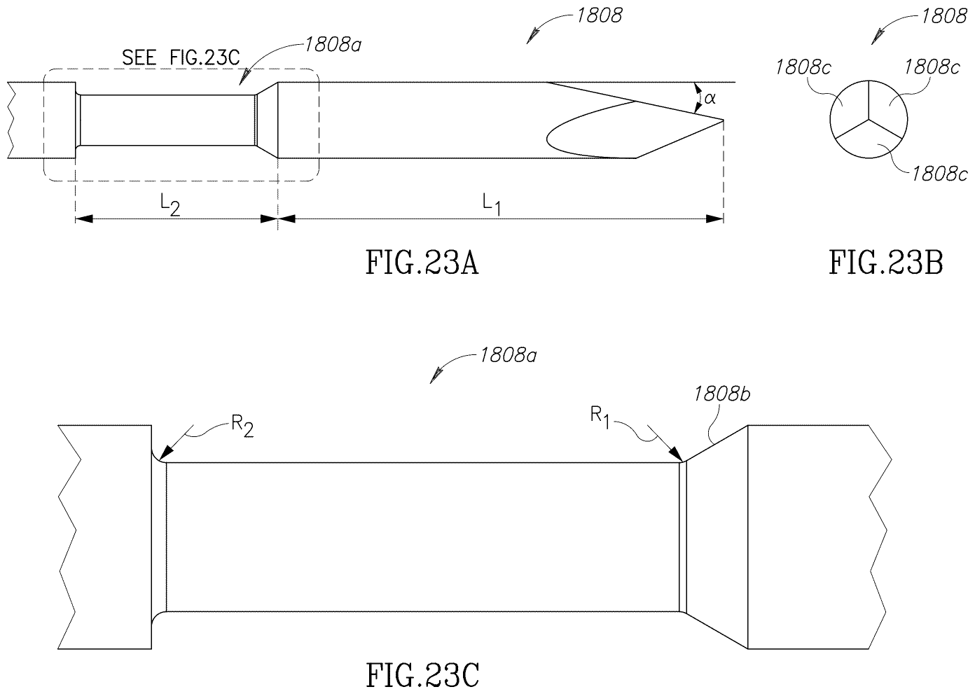

Reference is now made to FIG. 23A, which shows a side view of a distal area of needle 1808. In general, needle 1808 may be solid (not hollow) and made of a superelastic material such as Nitinol, or an elastic material such as stainless steel, CoCr, or the like. Needle 1808 may have a length similar to that of outer shaft 1802, such that it extends all the way to the handle. Needle 1808 may have a diameter of 0.4 to 1.5 mm (or alternatively beyond that), for example 0.4-0.7 mm, 0.5-0.8 mm, 0.6-0.9 mm, 0.7-1.0 mm, 0.8-1.1 mm, 0.9-1.2 mm, 1.0-1.3 mm, 1.1-1.4 mm, or 1.2-1.5 mm, except for a certain groove as discussed further below.

Needle 1808 may have a sharp end, for example a multi-bevel spear. FIG. 23B illustrates such sharp end from the front, where three exemplary bevels 1808c are shown. Each of bevels 1808c may have an angle (α) of 8-30 degrees (or alternatively beyond that) relative to the longitudinal axis of needle 1808, such as 8-12 degrees, 10-14 degrees, 12-16 degrees, 14-18 degrees, 16-20 degrees, 18-22 degrees, 20-24 degrees, 22-26 degrees, 24-28 degrees, or 26-30 degrees.

Needle 1808 may include a groove 1808a in its distal area, as shown in greater detail in FIG. 23C. Groove 1808a may be configured to receive an inward tab of the anchor, as will be discussed below in greater detail, such that the anchor remains secured over needle 1808 as the needle penetrates the tissue. Groove 1808a may have a length (L₂) of 1-3 mm (or alternatively beyond that), for example 1-2 mm, 1.5-2.5 mm, or 2-3 mm. Groove 1808a may have a diameter, at its thinnest area, of approximately 50-80% of the diameter of needle 1808 as discussed above. At its distal side, groove 1808a may include an inclined surface 1808b disposed at an angle of 15-45 degrees (or alternatively beyond that) relative to the longitudinal axis of needle, such as 15-25 degrees, 20-30 degrees, 25-35 degrees, 30-40 degrees, or 35-45 degrees. Optionally, a concave surface (R₁) may lie between inclined surface 1808b and the thinnest area of groove 1808a, so that a leading edge of the anchor's inward tab can slide outside the groove more easily; the concave surface (R₁) may have a radius of curvature of 0.02 to 0.10 mm, for example. At its proximal side, groove 1808a may include another concave surface (R₂) having a radius of curvature of 0.02 to 0.30 mm, for example. Generally speaking, groove 1808a may gradually reduce in diameter from the maximal diameter of needle 1808 to the thinnest area of the groove.

A tip area of needle 1808, from the needle's distal end to the beginning of groove 1808a, may have a length (L₁) of 1-10 mm (or alternatively beyond that), such as 1-3 mm, 2-4 mm, 3-5 mm, 4-6 mm, 5-7 mm, 6-8 mm, 7-9 mm, or 8-10 mm.

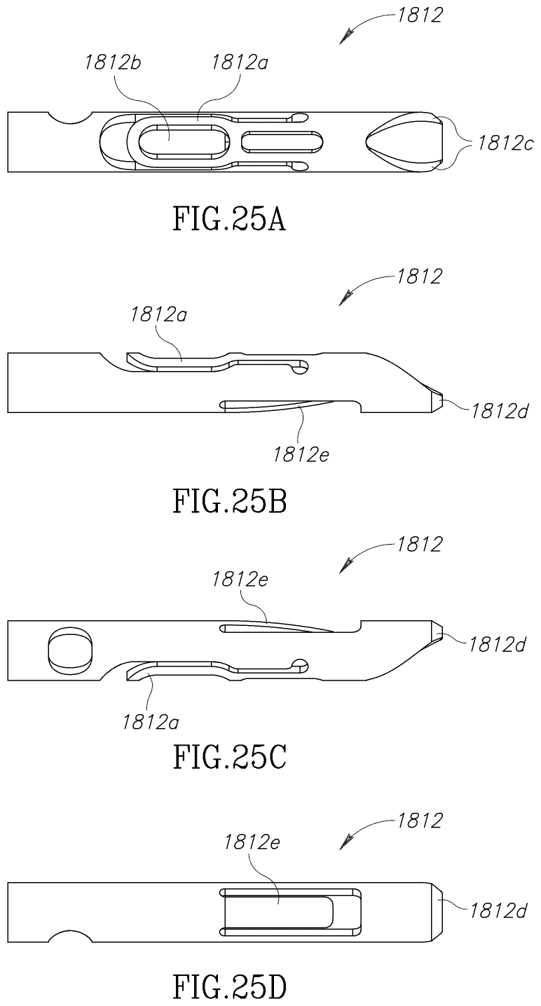

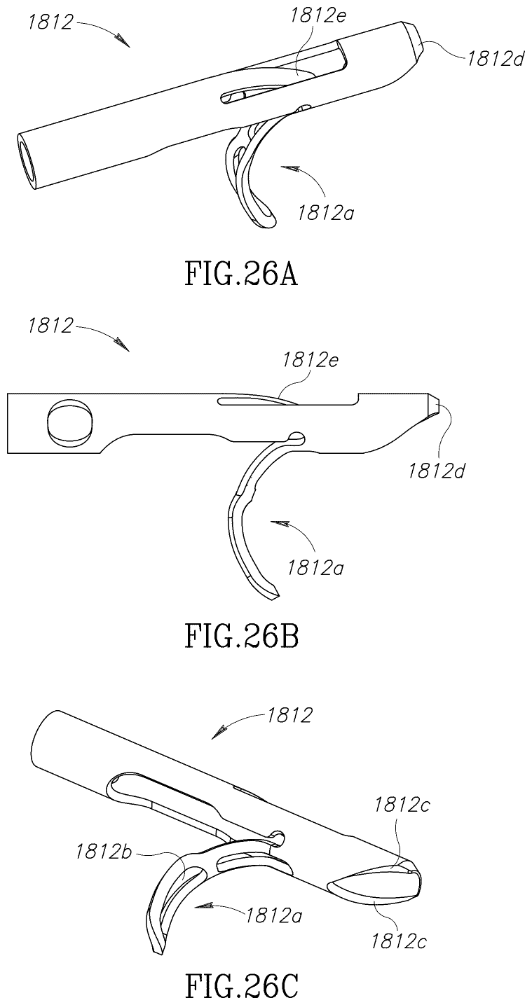

Reference is now made to FIGS. 25A-25D, which show anchor 1812 in four perpendicular side views, when the anchor is in a closed (biased) configuration. Reference is also made to FIGS. 26A-26C, which show anchor 1812 in a first perspective view, a side view, and a second perspective view, when the anchor is in an open (expanded, unbiased) configuration.

Anchor 1812 may have a generally tubular body, optionally cut (e.g., laser cut) from a tube of material or cut (e.g., laser cut) and rolled from a sheet of material; the material may be superelastic such as Nitinol, or elastic (and optionally resilient) such as stainless steel, CoCr, PEEK, or the like. The tubular body may have a wall thickness of approximately 0.1 to 0.4 mm (or alternatively beyond that), such as 0.1-0.2 mm, 0.15-0.25 mm, 0.2-0.3 mm, 0.25-0.35 mm, or 0.3-0.4 mm. The tubular body may have a length of approximately 4 to 16 mm (or alternatively beyond that), for example 4-7 mm, 6-9 mm, 8-11 mm, 10-13 mm, or 12-16 mm. The tubular body may have an outer diameter of approximately 0.5 to 2 mm (or alternatively beyond that), for example 0.5-0.8 mm, 0.7-1.0 mm, 0.9-1.2 mm, 1.1-1.4 mm, 1.3-1.6 mm, 1.5-1.8 mm, or 1.7-2.0 mm.

Anchor 1812 may include an extension 1812a which is essentially a shaped cut out of its side wall, and trained (for example using suitable heat treatment, in the case of Nitinol) to erect at an angle of approximately 45 to 135 degrees (or alternatively beyond that) relative to the longitudinal axis of the anchor, such as 45-60 degrees, 55-70 degrees, 65-80 degrees, 75-90 degrees, 85-100 degrees, 95-110 degrees, 105-120 degrees, 115-130 degrees, or 125-135 degrees. Optionally, with extension 1812a expanded, anchor 1812 has an essentially T shape. Extension 1812a may have a length of 2-8 mm (or alternatively beyond that), such as between 2-3 mm, 3-4 mm, 4-5 mm, 5-6 mm, 6-7 mm, or 7-8 mm. Extension 1812a may have a width of 0.5-2 mm (or alternatively beyond that), such as between 0.5-0.8 mm, 0.7-1.0 mm, 0.9-1.2 mm, 1.1-1.4 mm, 1.3-1.6 mm, 1.5-1.8 mm, or 1.7-2.0 mm. When inside inner tube 1805 (of FIG. 18B), extension 1812a may be biased to approximately the same tubular circumference of the entire anchor 1812. The ratio between the lengths of the tubular body of anchor 1812 and extension 1812a may be, for example, between 1:0.3 and 1:0.7, or more specifically 1:0.3-1:05, 1:0.4-1:0.6, or 1:0.5-1:0.7.

Extension 1812a may include an aperture (also ‘loop’ or ‘ring’) 1812b to allow passage of a suture (not shown here), similar to the previously-discussed anchors. The internal rim of aperture 1812b may be treated, such as by electropolishing, to be extremely smooth (for example, to a surface roughness of up to 10 or 20 microns) so that it does not damage the suture during tensioning. Additionally or alternatively, the internal rim may be coated with a smooth material, optionally polymeric.

Anchor 1812 may also include an inward tab 1812e which is cut out of its side wall, and trained (for example using suitable heat treatment) to protrude by approximately 0.1-1.3 mm into the void of the tubular body of the anchor. Inward tab 1812e may be disposed opposite extension 1812a.

An alternative anchor (not shown) may be similar to anchor 1812 but devoid of an extension. Instead, the suture may be threaded through a pair of apertures in a side wall of the anchor around its middle, or through any other suitable structure which does not greatly protrude from the anchor's side wall.

With reference back to FIG. 18B, it can be seen how the inward tab fits into the groove in needle 1808, so that anchor 1812 cannot slide distally and fall off the needle unless sufficient force is applied, such as a force of 0.1 to 1 kg (or alternatively beyond that), or more specifically 0.1-0.3 kg, 0.2-0.4 kg, 0.3-0.5 kg, 0.4-0.6 kg, 0.5-0.7 kg, 0.6-0.8 kg, 0.7-0.9 kg, or 0.8-1.0 kg. This force may be applied by a withdrawal of needle 1808 proximally, concurrently with holding anchor backer 1803 stationary, such that anchor 1812 slides distally over and off the needle.

Back to FIGS. 25A-25D and 26A-26C, anchor 1812 is optionally beveled 1812c and/or chamfered 1812d at its distal end, to minimize resistance from the tissue as the tissue is being punctured by needle 1808 and then the puncture being expanded by the passing anchor. Bevel 1812c and/or chamfer 1812d may each be at an angle of 10 to 50 degrees relative to the longitudinal axis of anchor 1812, such as 10-20 degrees, 15-25 degrees, 20-30 degrees, 25-35 degrees, 30-40 degrees, 35-45 degrees, or 40-50 degrees.

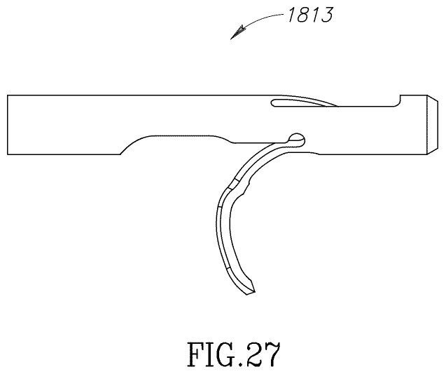

With reference to FIG. 27, an alternative anchor 1813 may be identical to anchor 1812 except it may lack a bevel at its distal end.

With reference back to FIGS. 18A-18B, anchor 1812 may supported from the back by anchor backer 1803, which is a tube optionally extending until the handle. Anchor backer 1803 may be made of one of the elastic or superelastic materials mentioned above, or a similar material. The outer diameter of anchor backer 1803 may be larger than the inner diameter of anchor 1812, such that the anchor is prevented from sliding backwards when the anchor backer is held stationary. This way, when needle 1808 is withdrawn proximally, anchor 1812 is forced to slide forward over the needle and is ultimately released from the needle.

With reference back to FIGS. 18A-18B, grasper collector 1804 may be a tube made of stainless steel, Co—Cr, or the like, and fixedly mounted over outer shaft 1802, for example using an adhesive or by melting the polymeric (e.g., PEEK) outer shaft onto the grasper collector. Grasper collector 1804 may have a series of optional apertures 1804b in its proximal 1-10 mm (or alternatively beyond that) to reinforce the interconnection between the grasper collector and outer shaft 1802, by allowing some of the adhesive to enter the apertures and dry or cure in them; this may further prevent the grasper collector from detaching and freely rolling over the outer shaft.

Grasper collector 1804 may further include one or more elongated slits 1804c disposed at an angle of 30-80 degrees relative to the longitudinal axis of the grasper collector and located inside the middle third of its length, to endow the grasper collector with certain axial flexibility; that is, when the grasper collector is inside the endoscope's work channel and the endoscope is articulated at acute angles, the flexibility of the grasper collector may prevent it from becoming stuck inside the work channel.

Grasper collector 1804 may further include stopper ring 1804a which was briefly mentioned above. Stopper ring 1804a may be made of one of the materials mentioned above with respect to grasper collector 1804, and may be configured to prevent grasper 1806 from accidentally sliding into outer tube 1802 when the grasper is withdrawn backwards; this might happen if the outer tube is polymeric (e.g., make of PEEK) and the superior rigidity of the backwards-moving grasper causes it to forcibly expand the inner diameter of the polymeric outer tube and become stuck therein. Stopper ring 1804a, being at least equally rigid to grasper 1806, may prevent this from happening.

As to outer shaft 1802, its purpose may be to enclose the more inner parts of shaft arrangement 1800. Outer shaft 1802 may have an outer diameter allowing it to fit within the endoscope's work channel, such as an outer diameter of up to 3 mm (or, in other embodiments, a larger outer diameter). Outer shaft 1802 may be made of a material such as stainless steel, PEEK, or the like. To increase its flexibility and ability to articulate within the flexible endoscope, outer shaft may be alternatively structured as a coil or a mesh.

An apparatus for implanting anchors beyond tissue in order to suture the tissue (the apparatus including shaft arrangement 1800 of FIGS. 18A-18B), may generally operate as follows (similarly to what has been described above with reference to FIGS. 16A-16D, except for certain differences which will become apparent from the following discussion):

The distal area of shaft arrangement 1800, with all its internal components, is brought adjacent to or in contact with the inner wall of the tissue. Shaft arrangement 1800 may be disposed, for example, inside a work channel of an endoscope (not shown), such as a gastroscope or a colonoscope, with only a distal portion of the shaft arrangement (e.g., its distalmost 5-30 mm) exposed beyond the work channel's distal end.

Inner tube 1805 is pushed distally and moved together with its attached grasper 1806, so that tines 1806b penetrate the tissue as they curl backwards, and grasp the tissue securely. As mentioned above, the penetration may be to a partial thickness or to the full thickness of the tissue.

Then, inner tube 1805 with grasper 1806 (also with outer shaft 1802 and grasper collector 1804) are pulled proximally, pulling the grasped tissue with it, while maintaining needle 1808, anchor 1812, and anchor backer 1803 stationary, in place. This creates the aforementioned pocket in the tissue, and simultaneously causes the sharp end of needle 1808, and subsequently also anchor 1812, to penetrate the full thickness of the tissue so that the anchor is fully within the pocket.

Next, needle 1808 is withdrawn proximally back into inner tube 1805 while maintaining anchor backer 1803 stationary, in place, so that anchor 1812 slides over the needle. Anchor 1812 is thus released and remains at the distal side of the tissue, inside the pocket.

Then, grasper 1806 may be released from the tissue by pulling it back into grasper collector 1804 (or directly into outer shaft 1802, if no grasper collector is used) or by advancing grasper collector 1804 (or outer shaft 1802) over the grasper, such that tines 1812b assume their biased, straight configuration.

Another anchor may then be loaded into shaft assembly 1800, to its designated location over the groove in needle 1808, and the procedure above repeated.

After all anchors have been implanted, the suture (not shown) may be tensioned so as to bring all anchors (and the tissue which they secure) closer together, and the suture may be secured relative to the last (most proximal) anchor, for example using a manual knot onto the anchor, or a cinch that is large enough so that it prevents the suture from sliding through the loop of the last anchor.

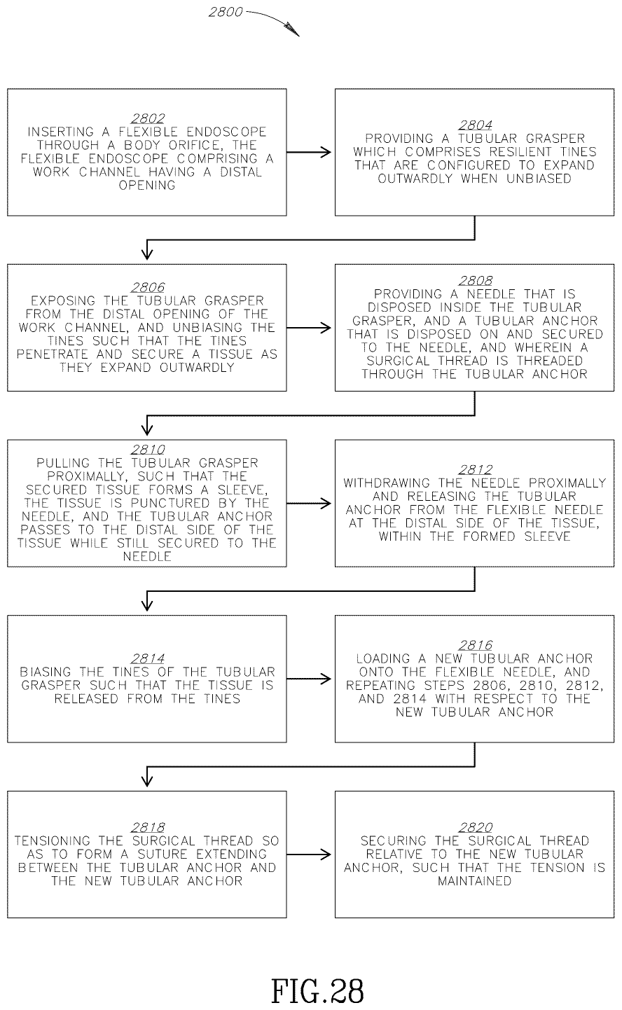

The manner of operation of the apparatus may be better understood with reference to FIG. 28, which is a flow chart of a method 2800 for suturing tissue, including the following steps:

A step 2802 may include inserting a flexible endoscope through a body orifice of a patient (for example, the mouth, nose, rectum, or vagina), the flexible endoscope comprising a work channel having a distal opening.

A step 2804 may include providing a tubular grasper which comprises resilient tines that are configured to expand outwardly when unbiased.

A step 2806 may include exposing the tubular grasper from the distal opening of the work channel, and unbiasing the tines such that the tines penetrate and secure a tissue as they expand outwardly.

A step 2808 may include providing a needle that is disposed inside the tubular grasper, and a tubular anchor that is disposed on and secured to the needle, and wherein a surgical thread is threaded through the tubular anchor.

A step 2810 may include pulling the tubular grasper proximally, such that the secured tissue forms a sleeve, the tissue is punctured by the needle, and the tubular anchor passes to the distal side of the tissue while still secured to the needle.

A step 2812 may include withdrawing the needle proximally and releasing the tubular anchor from the flexible needle at the distal side of the tissue, within the formed sleeve.

A step 2814 may include biasing the tines of the tubular grasper such that the tissue is released from the tines.

A step 2816 may include loading a new tubular anchor onto the flexible needle, and repeating steps 2806, 2810, 2812, and 2814 with respect to the new tubular anchor.

A step 2818 may include tensioning the surgical thread so as to form a suture extending between the tubular anchor and the new tubular anchor.

A step 2820 may include securing the surgical thread relative to the new tubular anchor, such that the tension is maintained.

An important aspect of embodiments of the invention is the layered, substantially concentric configuration of elements of the described shaft arrangements, each being generally tube-shaped and mounted over, behind, or in front of one or more of the other elements. This enables the suturing of tissue (by implanting a series of anchors interconnected by a suture) through a work channel of an endoscope, which only permits motion along the work channel's longitudinal axis; this axial motion of the elements (in relation to each other and to the work channel) prevents the need for various ‘overtube’ type tools that have to be mounted over the distal area of the endoscope. This also prevents the need to implement certain miniature, complex, and delicate tools that suture the tissue from side to side, namely-thread a suture in a direction substantially perpendicular to the endoscope's work channel.

Another important aspect of embodiments of the invention is the extension of the anchor, which can be biased to confirm to the generally tubular shape of the anchor once inside the pertinent shaft arrangement, and erects to an expanded configuration once the anchor is released. When the extension is expanded, such as to a perpendicular configuration relative to the longitudinal axis of the anchor's tubular body, it prevents the anchor from returning back to the proximal side of the tissue through the aperture previously created by the needle; namely, a T-shaped structure (anchor with expanded extension) is nearly impossible to pass through an aperture of a diameter similar to that of one of the T's arms. Optionally, when the suture is tensioned, only the end region of the extension, where the loop is disposed, returns through the aperture in the tissue.

A further important aspect of embodiments of the invention is that the grasper (such as the grasper of FIGS. 15E-15G, 18A-B, or others), and especially its tines, is configured to either penetrate the tissue only partially (not to its full thickness) when grasping it, or, in rarer cases, penetrate to the distal side of the tissue only once the tines have already expanded to an angle of more than approximately 60°, 70°, 80°, 90°, 100°, 110°, or 120° relative to the longitudinal axis of the grasper's tubular base, such that the distal edges of the times are less likely to damage any organs that happen to be adjacent to the penetrated tissue, on its distal side.

The needle may be hollow in some embodiments and solid in others, regardless of whether a needle of one type or another is described above, by way of example, in relation to a specific embodiment. Merely as an example, while needle 1808 of FIGS. 18A-18B is shown and discussed as a solid needle, it may just as well be structured as a hollow needle.

In some embodiments, a biocompatible lubricant may be introduced between the surfaces of the substantially concentric elements, to smoothen their sliding one over the other. For example, a lubricant may be provided between the outer shaft and the inner shaft (on which the grasper is mounted), between the inner shaft and the anchor backer, between the anchor backer and the needle, and/or the like. The lubricant may be introduced between such parts during fabrication of the apparatus, and/or during use of the apparatus by pumping the lubricant through a suitable opening in the handle, which disperses it to the various lumens. Instead of or in addition to a lubricant, a biocompatible washing fluid may be pumped through one or more of the lumens, in the distal direction, during use of the apparatus, to push and evacuate any dirt that may have entered the distal area of the shaft arrangement, such as blood, tissue particles, etc.

In some embodiments, each tubular element (or an element having a tubular portion) may have an inner diameter larger by 0.1 mm to 1.0 mm (for example, 0.1-0.3 mm, 0.2-0.4 mm, 0.3-0.5 mm, 0.4-0.6 mm, 0.5-0.7 mm, 0.6-0.8 mm, 0.7-0.9 mm, or 0.8-1.0 mm) than the outer diameter of the tubular element (or an element having a tubular portion) disposed inside it. This spacing between the overlayed (and optionally, substantially concentric) elements facilitates their relative motion (sliding one over the other) but is also small enough so that the outermost element, such as the aforementioned outer shaft (and/or grasper collector) has a small enough outer diameter to fit within a work channel of an endoscope.

The handle in embodiments of the invention may include multiple actuators that are physically connected at least some of the elements of the shaft arrangement, that enable a user (such as a surgeon) to manipulate these elements from the handle, to effect the various steps of suturing the tissue as described above. Alternatively, such actuators may be provided without a handle.

During use of some embodiments of the invention in a patient's stomach (from an esophageal approach of the endoscope), the tissue (stomach wall) sleeve that is formed by pulling back the grasper may assume a substantially cylindrical shape and not (or substantially not) a conical shape, because the abdominal cavity is typically in a vacuum state during surgery. It is also possible to operate a vacuum pump through the endoscope, such that the stomach shrinks over the distal area of the endoscope (and over whatever parts of the shaft arrangement that extends out of the distal end of the endoscope), thereby more conveniently facilitating the operation of the shaft arrangement to suture the tissue, and especially the grasping of the tissue using the grasper.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims, each of the terms “substantially,” “essentially,” and forms thereof, when describing a numerical value, means up to a 20% deviation (namely, ±20%) from that value; similarly, when such a term describes a numerical range, it means an up to 20% broader range (10% on each side of the range). Additionally, when each of the terms “substantially,” “essentially,” and forms thereof is used to describe a geometrical term defining an angle (such as “perpendicular,” “orthogonal,” “parallel,” “planar,” “co-planar,” “co-axial,” “horizontal,” “vertical,” etc.), it means up to a 25 degrees deviation (namely,) ±25° from that angle.

The description of a numerical range should be considered to have specifically disclosed all the possible subranges as well as individual numerical values within that range. For example, description of a range from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Similarly, description of a range of fractions from 0.6 to 1.1 should be considered to have specifically disclosed subranges such as from 0.6 to 0.9, from 0.7 to 1.1, from 0.9 to 1, from 0.8 to 0.9, from 0.6 to 1.1, from 1 to 1.1 etc., as well as individual numbers within that range, for example, 0.6, 0.7, 0.8, 0.9, 1, and 1.1.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. An apparatus comprising:
a tubular grasper comprising resilient tines that are configured to expand outwardly when unbiased;
a needle disposed inside the tubular grasper, and configured to penetrate tissue;
a tubular anchor disposed on the needle; and
a surgical thread threaded through the tubular anchor.

2. The apparatus of claim 1, further comprising one or more actuators configured to:
push the tubular grasper distally, out of a distal end of an endoscope, such that the tines expand outwardly, penetrate tissue, and secure the tissue to the grasper;
pull the tubular grasper proximally, such that the tissue that is secured to the grasper is pulled proximally while the needle and the tubular anchor penetrate the tissue so as to position the anchor at the distal side of the tissue;
pull the flexible needle proximally and release the tubular anchor beyond the tissue; and
tension the surgical thread at the proximal side of the tissue, thereby causing the tubular anchor to pull the tissue proximally.

3. The apparatus of claim 2, wherein the one or more actuators are comprised at least partially in a handle.

4. The apparatus of claim 1, wherein the tubular anchor comprises a tubular body and a resilient extension that is configured to expand outwardly from the tubular body when unbiased, and wherein the extension comprises a ring through which the surgical thread is threaded.

5. The apparatus of claim 1, further comprising:
an inner shaft to which the tubular grasper is affixed; and
a flexible, tubular, outer shaft configured to enclose the inner shaft.

6. The apparatus of claim 5, wherein the one or more actuators are comprised at least partially in a handle, and wherein the inner shaft extends to the handle.

7. The apparatus of claim 1, further comprising:
a tubular grasper collector mounted over the outer shaft and configured to enclose the tubular grasper.

8. The apparatus of claim 1, wherein each of the tines of the tubular grasper has a blunt edge.

9. The apparatus of claim 1, wherein the extension is a cut through the wall of the tubular anchor.

10. The apparatus of claim 4, wherein a length ratio between the tubular body of the anchor and the extension of the anchor is between 1:0.3 and 1:0.7.

11. The apparatus of claim 1, wherein the tubular body of the anchor has a chamfered distal edge to facilitate penetration of the tissue together with the needle.

12. The apparatus of claim 1, wherein the tubular body of the anchor has a beveled distal edge to facilitate penetration of the tissue together with the needle.

13. (canceled)

14. (canceled)

15. (canceled)

16. (canceled)

17. (canceled)

18. (canceled)

19. A method comprising:
a) inserting a flexible endoscope through a body orifice of a patient, the flexible endoscope comprising a work channel having a distal opening;
b) providing a tubular grasper which comprises resilient tines that are configured to expand outwardly when unbiased;
c) exposing the tubular grasper from the distal opening of the work channel, and unbiasing the tines such that the tines penetrate and secure a tissue as they expand outwardly;
d) providing a needle that is disposed inside the tubular grasper, and a tubular anchor that is disposed on and secured to the needle, and wherein a surgical thread is threaded through the tubular anchor;
e) pulling the tubular grasper proximally, such that the secured tissue forms a sleeve, the tissue is punctured by the needle, and the tubular anchor passes to the distal side of the tissue while still secured to the needle;
f) withdrawing the needle proximally and releasing the tubular anchor from the flexible needle at the distal side of the tissue, within the formed sleeve;
g) biasing the tines of the tubular grasper such that the tissue is released from the tines;
h) loading a new tubular anchor onto the flexible needle, and repeating steps c, e, f, and g with respect to the new tubular anchor;
i) tensioning the surgical thread so as to form a suture extending between the tubular anchor and the new tubular anchor; and
j) securing the surgical thread relative to the new tubular anchor, such that the tension is maintained.

20. The method of claim 19, further comprising providing one or more actuators configured to facilitate steps c, e, f, and g.

21. The method of claim 20, wherein the one or more actuators are comprised at least partially in a handle.

22. The method of claim 19, wherein the tubular anchor comprises a tubular body and a resilient extension that is configured to expand outwardly from the tubular body when unbiased, and wherein the extension comprises a ring through which the surgical thread is threaded.

23. The method of claim 19, further comprising:
providing an inner shaft to which the tubular grasper is affixed; and
providing a flexible, tubular, outer shaft configured to enclose the inner shaft.

24. The method of claim 23, wherein the one or more actuators are comprised at least partially in a handle, and wherein the inner shaft extends to the handle.

25. The method of claim 19, further comprising:
providing a tubular grasper collector mounted over the outer shaft and configured to enclose the tubular grasper.

26. The method of claim 19, wherein each of the tines of the tubular grasper has a blunt edge.

27. The method of claim 19, wherein the extension is a cut through the wall of the tubular anchor.

28. The method of claim 22, wherein a length ratio between the tubular body of the anchor and the extension of the anchor is between 1:0.3 and 1:0.7.

29. The method of claim 22, wherein the tubular body of the anchor has a chamfered distal edge to facilitate penetration of the tissue together with the needle.

30. The method of claim 22, wherein the tubular body of the anchor has a beveled distal edge to facilitate penetration of the tissue together with the needle.
